(19) United States
(12) Patent Application Publication
(10) Pub. No.: US 2024/0210344 A1
(43) Pub. Date: Jun. 27, 2024

(54) DISPLAY APPARATUS AND SENSING METHOD USING THE SAME

(71) Applicants: Samsung Display Co., LTD., Yongin-si (KR); Pusan National University Industry-University Cooperation Foundation, Busan (KR)

(72) Inventors: Jiwon LEE, Yongin-si (KR); Jinwoo OH, Busan (KR); Eunjung CHOI, Busan (KR); Jangyeol YOON, Yongin-si (KR); Jongho HONG, Yongin-si (KR)

(21) Appl. No.: 18/545,982

(22) Filed: Dec. 19, 2023

(30) Foreign Application Priority Data
Dec. 21, 2022 (KR) 10-2022-0181069

(51) Int. Cl. G01N 27/12; G01N 33/543; G01N 33/98

(52) U.S. Cl. CPC G01N 27/128; G01N 33/5438; G01N 33/98; G01N 2333/01; G01N 2800/12

(57) ABSTRACT

A display apparatus includes: a substrate including a sensor area and a display area, a plurality of biosensors disposed on the substrate and of which electrical resistance changes when exposed to a target material, and an organic insulating layer defining a plurality of openings therein, which expose the plurality of biosensors to the outside, where the display area includes a plurality of first through-portions, and a plurality of display portions spaced apart from each other by the plurality of first through-portions, and the sensor area includes a plurality of second through-portions, and a plurality of base portions spaced apart from each other by the plurality of second through-portions.

This application claims to Korean Patent Application No. 10-2022-0181069, filed on Dec. 21, 2022, and all the benefits accruing therefrom under 35 U.S.C. § 119, the content of which in its entirety is herein incorporated by reference.

### BACKGROUND

### 1. Field

One or more embodiments relate to a structure of a display apparatus and a sensing method using the display apparatus.

### 2. Description of the Related Art

A display apparatus visually displays data. The display apparatus may include a substrate partitioned into a display area and a peripheral area. The display area may include scan lines and data lines insulated from each other and may include a plurality of pixels. Also, a thin-film transistor and a subpixel electrode electrically connected to the thin-film transistor may be provided in the display area to correspond to each of the pixels. In addition, an opposite electrode commonly provided to the pixels may be provided in the display area. Various wires, a scan driver, a data driver, a controller, a pad portion, and the like may be provided in the peripheral area to transmit electrical signals to the display area.

The display apparatus has been used for various purposes. Accordingly, various attempts have been made to design to improve the quality of the display apparatus.

### SUMMARY

One or more embodiments provide a flexible and stretchable display apparatus capable of easily testing respiratory diseases or the like. However, the embodiments are examples, and do not limit the scope of the disclosure.

Additional aspects will be set forth in part in the description which follows and, in part, will be apparent from the description, or may be learned by practice of the presented embodiments.

According to one or more embodiments, a display apparatus: includes a substrate including a sensor area and a display area, a plurality of biosensors disposed on the substrate and of which electrical resistance changes when exposed to a target material, and an organic insulating layer defining a plurality of openings therein, which expose the plurality of biosensors to the outside, where the display area includes a plurality of first through-portions, and a plurality of display portions spaced apart from each other by the plurality of first through-portions, and the sensor area includes a plurality of second through-portions, and a plurality of base portions spaced apart from each other by the plurality of second through-portions.

According to an embodiment, each of the plurality of biosensors may include a first electrode, a second electrode, and a first coating layer, which connects the first electrode to the second electrode.

According to an embodiment, the first electrode and the second electrode may be arranged on substantially the same layer as a source electrode and a drain electrode of a thin-film transistor arranged in the plurality of display portions.

According to an embodiment, the plurality of openings may allow the first coating layer to be in contact with external air.

According to an embodiment, the first coating layer may include an M13 bacteriophage.

According to an embodiment, the plurality of biosensors may each be an electrical sensor in which, when exposed to the target material, a surface protein of the M13 bacteriophage swells and an electron movement path changes.

According to an embodiment, the M13 bacteriophage may be provided in plurality, and the plurality of biosensors may include the M13 bacteriophages genetically modified differently from each other, respectively.

According to an embodiment, a surface protein of each of the genetically-modified M13 bacteriophages may include a peptide having a sequence in which at least one of amino acids is consecutively arranged.

According to an embodiment, the display area may further include a plurality of connection portions extending in different directions from the plurality of display portions, the sensor area may further include a plurality of bridge portions extending in different directions from the plurality of base portions, at least one of the plurality of connection portions may be configured to connect two adjacent display portions among the plurality of display portions to each other, and at least one of the plurality of bridge portions may be configured to connect two adjacent base portions among the plurality of base portions to each other.

According to an embodiment, a distance between the two adjacent display portions among the plurality of display portions and a distance between the two adjacent base portions among the plurality of base portions may be changed by an external force, and the plurality of connection portions and the plurality of bridge portions may be stretchable by the external force.

According to an embodiment, the plurality of biosensors may include a plurality of first biosensors arranged in the plurality of base portions, respectively, and a plurality of second biosensors arranged in the plurality of bridge portions, respectively.

According to an embodiment, each of the plurality of base portions may include a sensor thin-film transistor, and a source electrode and a drain electrode of the sensor thin-film transistor may be used as metal electrodes of the plurality of first biosensors.

According to an embodiment, each of the plurality of bridge portions may have a structure in which the substrate, a first organic material layer disposed on the substrate, and the plurality of second biosensors disposed on the first organic material layer are stacked.

According to an embodiment, the plurality of biosensors may be arranged in the plurality of connection portions of the display area, respectively.

According to an embodiment, the substrate may further include a peripheral area surrounding the display area, and the sensor area may overlap the peripheral area.

According to one or more embodiments, an electronic apparatus includes a sensor unit. The sensor unit includes a substrate, a plurality of biosensors disposed on the substrate where each of the plurality of biosensors includes a first electrode, a second electrode, and a first coating layer connecting the first electrode to the second electrode, and an organic insulating layer defining a plurality of openings therein, which expose the plurality of biosensors to the outside, where the first coating layer includes an M13 bacteriophage, and when the sensor unit is exposed to a target material, electrical resistance of the plurality of biosensors changes.

According to an embodiment, the electronic apparatus may further include a display panel, and the sensor unit may be a separable external module electrically connected to the display panel for displaying an image, and the sensor unit may be detachable from the display panel.

According to an embodiment, the electronic apparatus may further include a display panel for displaying an image, where a sensor area and a display area may be defined in the display panel, and the sensor unit may be arranged in the sensor area.

According to an embodiment, the display area may include a plurality of first through-portions, a plurality of display portions spaced apart from each other by the plurality of first through-portions, and a plurality of connection portions extending in different directions from the plurality of display portions, the sensor area may include a plurality of second through-portions, a plurality of base portions spaced apart from each other by the plurality of second through-portions, and a plurality of bridge portions extending in different directions from the plurality of base portions, at least one of the plurality of connection portions may be configured to connect two adjacent display portions among the plurality of display portions to each other, and at least one of the plurality of bridge portions may be configured to connect two adjacent base portions among the plurality of base portions to each other.

According to an embodiment, the plurality of biosensors may include a plurality of first biosensors arranged in the plurality of base portions, respectively, and a plurality of second biosensors arranged in the plurality of bridge portions, respectively.

According to an embodiment, the first electrode and the second electrode may be arranged on substantially the same layer as a source electrode and a drain electrode of a thin-film transistor arranged in the plurality of display portions.

According to one or more embodiments, a sensing method of a display apparatus includes providing a display apparatus including a substrate, a plurality of biosensors, and an organic insulating layer, the substrate including a sensor area and a display area, the plurality of biosensors disposed on the substrate and of which electrical resistance changes when exposed to a target material, and the organic insulating layer defining a plurality of openings therein, which expose the plurality of biosensors to the outside, blowing an exhalation of a test subject into the sensor area, detecting, by the plurality of biosensors, an electrical signal based on a volatile organic compound (“VOC”) included in the exhalation of the test subject, and diagnosing whether a respiratory disease is present through the detected electrical signal.

According to an embodiment, the providing of the display apparatus may further include providing a sensor array with the plurality of biosensors each including an M13 bacteriophage genetically modified differently.

According to an embodiment, the detecting of the electrical signal may include detecting, by the genetically-modified M13 bacteriophage, the VOC included in the exhalation, allowing an electron movement path to change due to swelling of a surface protein of the M13 bacteriophage in response to the VOC, and calculating a changed electrical signal of each of the plurality of biosensors.

According to an embodiment, the diagnosing of the presence of the respiratory disease may include accumulating data about an electrical signal for each VOC, determining a type of the VOC by comparing the electrical signal for the VOC included in the exhalation of the test subject with the accumulated data, and determining whether the respiratory disease is present according to the type of the VOC included in the exhalation of the test subject.

According to an embodiment, the accumulating of the data about the electrical signal for each VOC may be performed by a statistical analysis method, such as hierarchical clustering analysis (“HCA”).

According to an embodiment, in the determining of the type of the VOC, the type of the VOC may be identified by a dendrogram analyzed through the HCA.

According to an embodiment, the sensing method may further include displaying, on a screen of the display apparatus, a result of the diagnosis.

### BRIEF DESCRIPTION OF THE DRAWINGS

The above and other aspects, features, and advantages of certain embodiments of the disclosure will be more apparent from the following description taken in conjunction with the accompanying drawings, in which:

FIGS. 1A to 1D are schematic diagrams of a display apparatus according to various embodiments;

FIG. 2 is a schematic plan view of a display apparatus according to an embodiment;

FIG. 3 is a schematic equivalent circuit diagram of a pixel circuit applicable to a display panel, according to an embodiment;

FIG. 4A is a schematic enlarged plan view of a region A of FIG. 2;

FIG. 4B is a plan view of a display panel of FIG. 4A stretched in a first direction and a second direction;

FIGS. 5A and 5B are schematic cross-sectional views of a display apparatus according to an embodiment;

FIG. 6 is a schematic cross-sectional view of the display panel taken along line II-II′ of FIG. 4A;

FIG. 7 is a schematic enlarged plan view of a region B of FIG. 2;

FIG. 8 is a schematic cross-sectional view of the display panel taken along lines III-III′ and IV-IV′ of FIG. 7;

FIG. 9 is an enlarged plan view of a portion of a display apparatus, according to another embodiment;

FIG. 10 is a schematic cross-sectional view of the display panel taken along line V-V′ of FIG. 9;

FIGS. 11A to 11E are diagrams for describing an M13 bacteriophage of a biosensor, according to an embodiment;

FIG. 12 is a flowchart of a sensing method using a display apparatus, according to an embodiment;

FIGS. 13A to 13G are diagrams for describing a sensing method using a biosensor, according to an embodiment; and

FIG. 14 is a schematic diagram of an electronic apparatus according to another embodiment.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, “a”, “an,” “the,” and “at least one” do not denote a limitation of quantity, and are intended to include both the singular and plural, unless the context clearly indicates otherwise. For example, “an element” has the same meaning as “at least one element,” unless the context clearly indicates otherwise. “At least one” is not to be construed as limiting “a” or “an.” “Or” means “and/or.” As used herein, the term “and/or” includes any and all combinations of one or more of the associated listed items. Throughout the disclosure, the expression “at least one of a, b or c” indicates only a, only b, only c, both a and b, both a and c, both b and c, all of a, b, and c, or variations thereof.

As various modifications may be applied and numerous embodiments may be implemented, particular embodiments will be illustrated in the drawings and described in detail in the written description. Effects and features, and methods for achieving them will be clarified with reference to embodiments described below in detail with reference to the drawings. However, the embodiments may have different forms and should not be construed as being limited to the descriptions set forth herein.

Hereinafter, the embodiments will now be described in detail with reference to the accompanying drawings. When described with reference to the drawings, identical or corresponding elements will be given the same reference numerals, and redundant description of these elements will be omitted.

While terms as “first,” “second,” etc., may be used to describe various elements, such elements are not to be limited to the above terms. The above terms are used only to distinguish one element from another.

“Substantially the same”, “about” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within +30%, 20%, 10% or 5% of the stated value.

It will be understood that terms, such as “comprise,” “include,” and “have,” used herein specify the presence of stated features or elements, but do not preclude the presence or addition of one or more other features or elements.

It will be further understood that, when a layer, region, or element is referred to as being “on” another layer, region, or element, it can be directly or indirectly on the other layer, region, or element. That is, e.g., intervening layers, regions, or elements may be present.

Sizes of elements in the drawings may be exaggerated for convenience of description. For example, because sizes and thicknesses of elements in the drawings are arbitrarily illustrated for convenience of description, the embodiments are not limited thereto.

When a certain embodiment may be implemented differently, a specific process order may be performed differently from the described order. For example, two processes that are successively described may be performed substantially simultaneously or performed in the order opposite to the order described.

In the following embodiments, it will be understood that when a layer, region, or element is referred to as being “connected to” or “coupled to” another layer, region, or element, it may be directly or indirectly connected or coupled to the other layer, region, or element. That is, e.g., intervening layers, regions, or elements may be present. For example, it will be understood that when a layer, region, or element is referred to as being “electrically connected to” or “electrically coupled to” another layer, region, and element, it may be directly or indirectly electrically connected or coupled to the other layer, region, or element. That is, e.g., intervening layers, regions, or elements may be present.

A display apparatus is an apparatus for displaying a moving image or a still image, and may be used as a display screen of not only portable electronic apparatuses, such as a mobile phone, a smartphone, a tablet personal computer (“PC”), a mobile communication terminal, an electronic notebook, an electronic book, a portable multimedia player (“PMP”), a navigation apparatus, and an ultra-mobile PC (“UMPC”), but also various products, such as a television, a laptop computer, a monitor, a billboard, and Internet of things (“IoT”). Also, a display apparatus according to an embodiment may be used for wearable apparatuses, such as a smart watch, a watch phone, a glasses-type display, and a head mounted display (“HMD”). In addition, a display apparatus according to an embodiment may be used as a panel of a vehicle, a center information display (“CID”) arranged on a center fascia or dashboard of a vehicle, a room mirror display replacing a side mirror of a vehicle, or a display arranged on a rear surface of a front seat, as entertainment for a back seat of a vehicle.

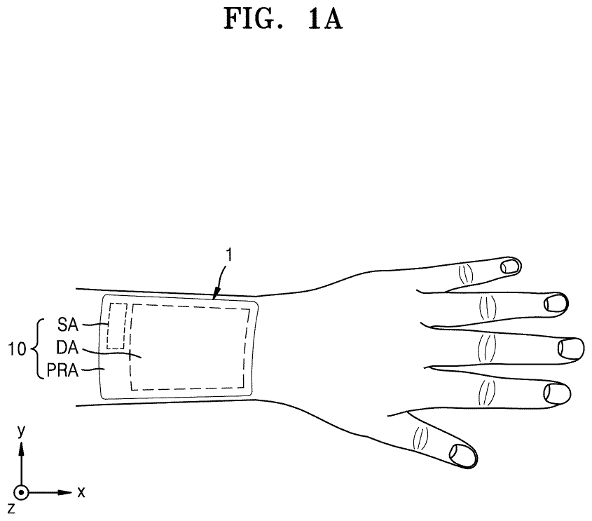

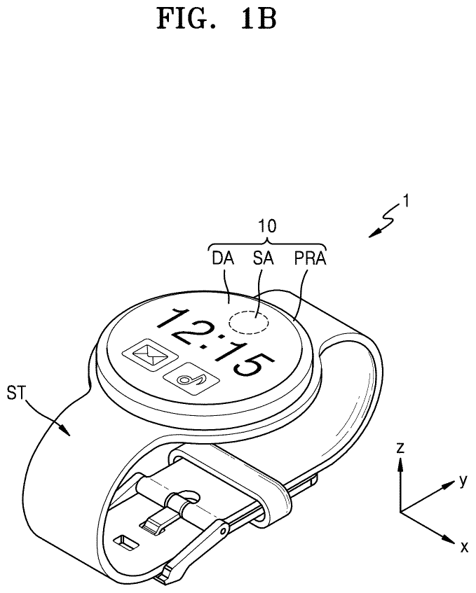

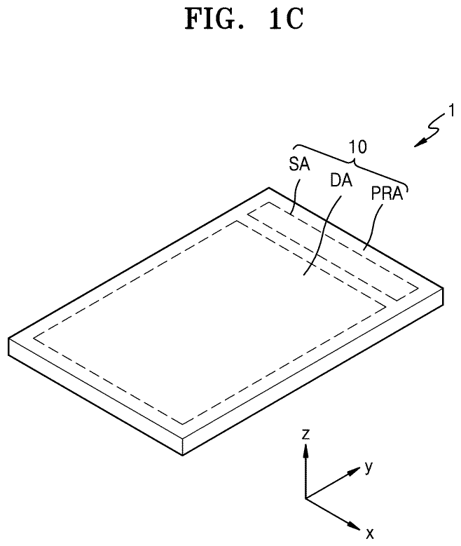

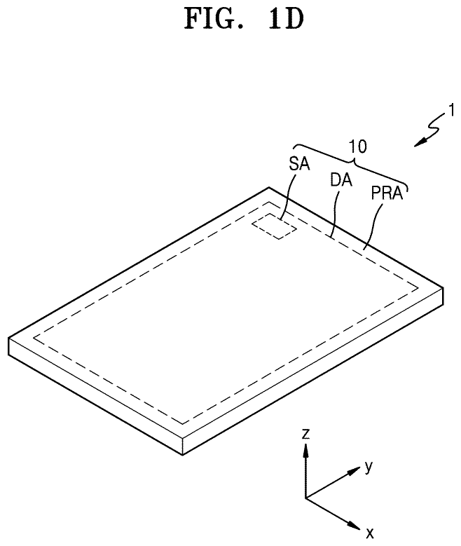

FIGS. 1A to 1D are schematic diagrams of a display apparatus 1 according to various embodiments.

The display apparatus 1 may display an image. In an embodiment, the display apparatus 1 may display diagnosis results for various diseases. In an embodiment, for example, the display apparatus 1 may sense a target material in a gas or liquid state emitted from a user, and display a diagnosis result of a disease suffered by the user from the target material. The target material may include a material included in a gas, such as breath or vaporized sweat, or a material included in a liquid, such as sweat, saliva, or tears. In another embodiment, the display apparatus 1 may display a diagnosis result of the user's physical condition. In an embodiment, for example, the display apparatus 1 may sense the target material in the gas or liquid state emitted from the user, and display the diagnosis result of the user's physical condition from the target material.

The display apparatus 1 may include a display panel 10. The display panel 10 may include a plurality of pixels (not shown) and display an image. The display panel 10 may include a display area DA, a sensor area SA, and a peripheral area PRA.

The plurality of pixels may be arranged in the display area DA. Accordingly, the display panel 10 may display an image in the display area DA with light emitted from the plurality of pixels.

The sensor area SA may be an area for detecting the target material in the gas or liquid state. In an embodiment, a plurality of biosensors of which electrical resistance changes when exposed to the target material may be arranged in the sensor area SA. In this case, a value of resistance or current flowing through the plurality of biosensor may be measured to identify whether the target material is present.

In an embodiment, the sensor area SA may have a polygonal shape. In another embodiment, the sensor area SA may have a circular or elliptical shape. Also, in another embodiment, the sensor area SA may have a curved edge.

The peripheral area PRA may be an area that does not provide an image. The peripheral area PRA may be an area in which the plurality of pixels are not arranged. However, a driver or the like for providing an electrical signal or power to the pixels may be arranged in the peripheral area PRA. In an embodiment, a controller (not shown) that determines whether the target material is present may be arranged in the peripheral area PRA. The controller may control the display panel 10 to display information about the presence of the target material. Accordingly, the display apparatus 1 may display the information about the presence of the target material.

In an embodiment, the peripheral area PRA may at least partially surround the display area DA and the sensor area SA. In an embodiment, for example, the peripheral area PRA may entirely surround the sensor area SA and the display area DA. That is, the sensor area SA may be arranged outside the display area DA. In another embodiment, the display area DA may at least partially surround the sensor area SA. In an embodiment, for example, the display area DA may entirely surround the sensor area SA. That is, the sensor area SA may also be arranged inside the display area DA.

Referring to FIG. 1A, the display apparatus 1 may be attached to a user. In an embodiment, the display apparatus 1 may include an adhesive layer and be attached to the user. In an embodiment, the display apparatus 1 may be modified into various shapes. In an embodiment, for example, the display apparatus 1 may be modified according to a surface shape of the user's skin.

Referring to FIG. 1B, the display apparatus 1 may be used in a wearable apparatus, such as a smart watch. In an embodiment, the display apparatus 1 may be coupled to a watch band ST.

Referring to FIGS. 1C and 1D, the display apparatus 1 may be used as a screen of a portable electronic apparatus, such as a mobile phone or a tablet PC. In an embodiment, as shown in FIG. 1C, the sensor area SA is arranged outside the display area DA, and accordingly, the peripheral area PRA may entirely surround the sensor area SA. In another embodiment, as shown in FIG. 1D, the sensor area SA is arranged inside the display area DA, and accordingly, the display area DA may entirely surround the sensor area SA, and the peripheral area PRA may entirely surround the display area DA.

In the following description, a case in which the display apparatus 1 is used as a screen of a portable electronic apparatus, such as a mobile phone or a tablet PC, as shown in FIGS. 1C and 1D, is mainly described in detail. However, the embodiment is not limited thereto, embodiments to be described below may be applied to various types of apparatuses including the display apparatus 1 of FIGS. 1A and 1B.

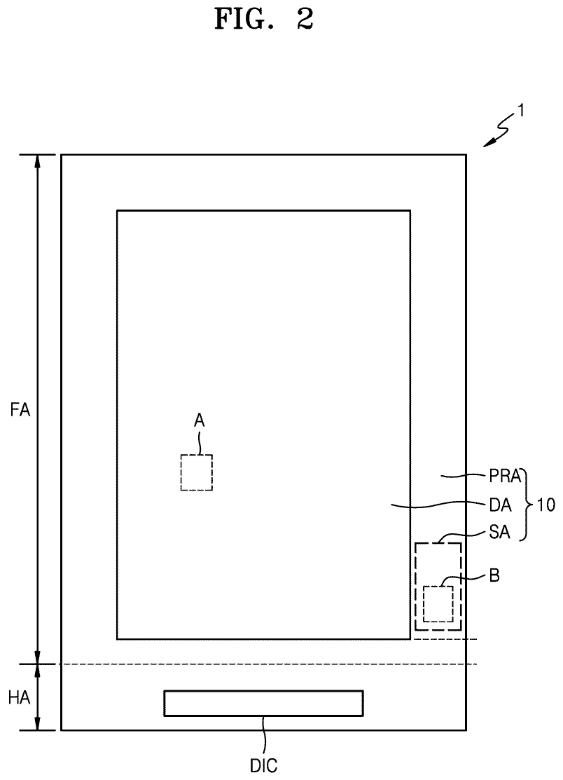

FIG. 2 is a schematic plan view of the display apparatus 1 according to an embodiment.

The display apparatus 1 may include the display panel 10. The display panel 10 may include the display area DA, the sensor area SA, and the peripheral area PRA. As described above, the display area DA may include a plurality of pixels (not shown). The display panel 10 may display an image by using the plurality of pixels. The peripheral area PRA may be an area in which the plurality of pixels are not arranged.

Each of the plurality of pixels may include a display element. The display panel 10 may include an organic light-emitting display panel using an organic light-emitting diode (“OLED”) including an organic emission layer. Alternatively, the display panel 10 may include a light-emitting diode (“LED”) display panel using an LED. A size of the LED may be on a micro scale or nano scale, and for example, the LED may include a micro LED. Alternatively, the LED may include a nanorod LED. The nanorod LED may include gallium nitride (GaN). According to an embodiment, a color conversion layer may be arranged over the nanorod LED. The color conversion layer may include quantum dots.

Alternatively, the display panel 10 may include a quantum dot light-emitting display panel using a quantum dot LED including a quantum dot emission layer. Alternatively, the display panel 10 may include an inorganic light-emitting display panel using an inorganic light-emitting element including an inorganic semiconductor. In the following description, a case in which the display panel 10 includes an organic light-emitting display panel that uses an organic LED as a display element is mainly described in detail.

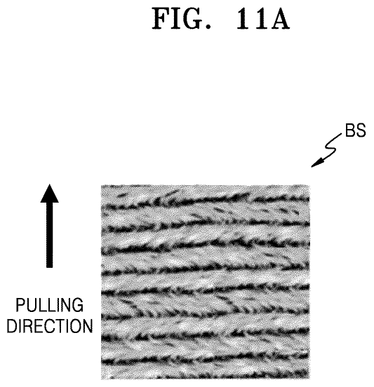

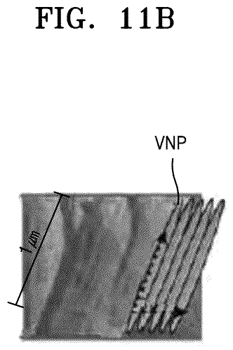

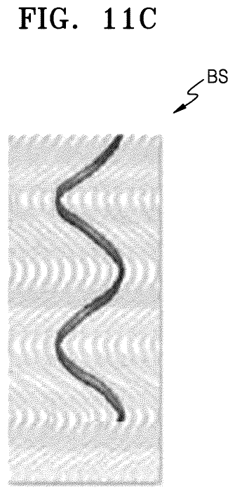

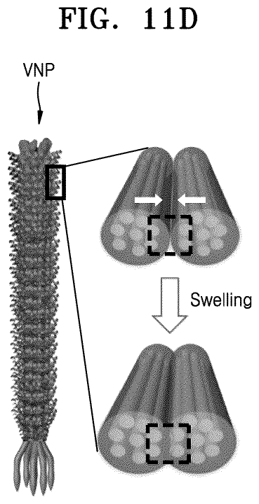

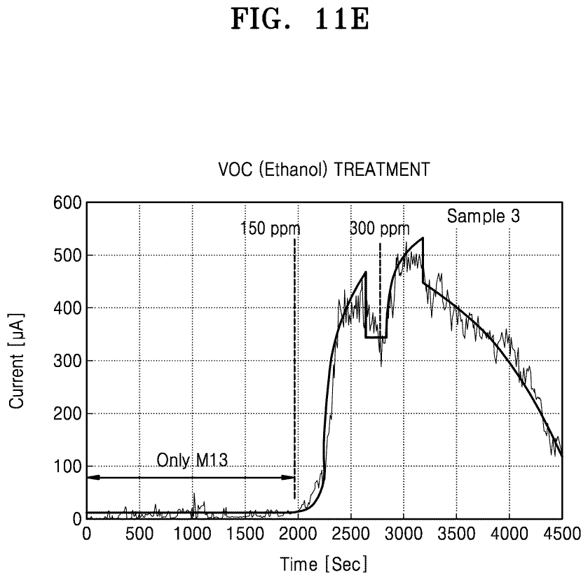

The sensor area SA may include a plurality of biosensors. In an embodiment, the plurality of biosensors may each include an M13 bacteriophage. When the M13 bacteriophage is exposed to a target material, a surface protein thereof swells and an electron movement path may change. Accordingly, the plurality of biosensors may detect electrical signal data differently according to the target material. The plurality of biosensors will be described in detail with reference to FIGS. 11A to 11E.

Also, a flexible area FA and a hard area HA may be defined in the display panel 10. The flexible area FA may refer to a relatively flexible area in the display panel 10, and the hard area HA may refer to a relatively hard area in the display panel 10.

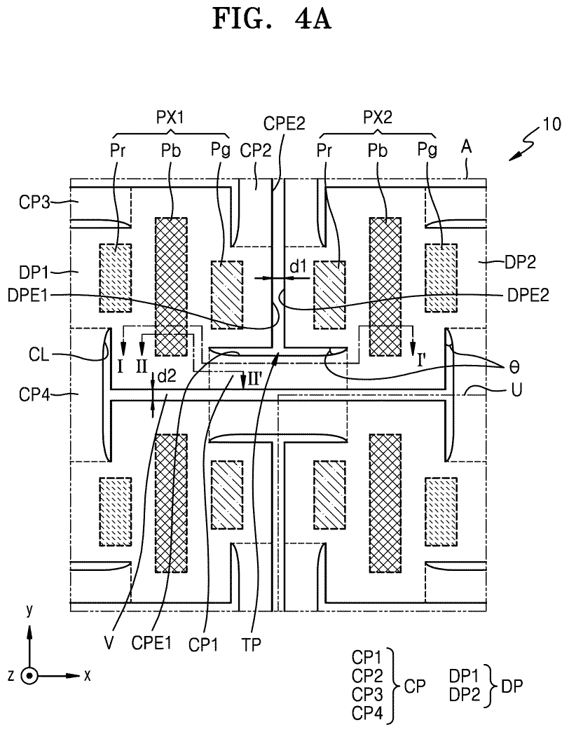

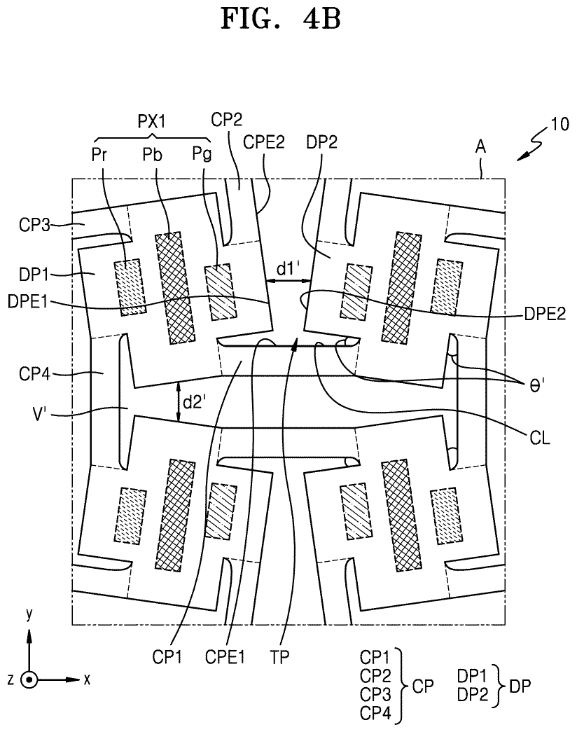

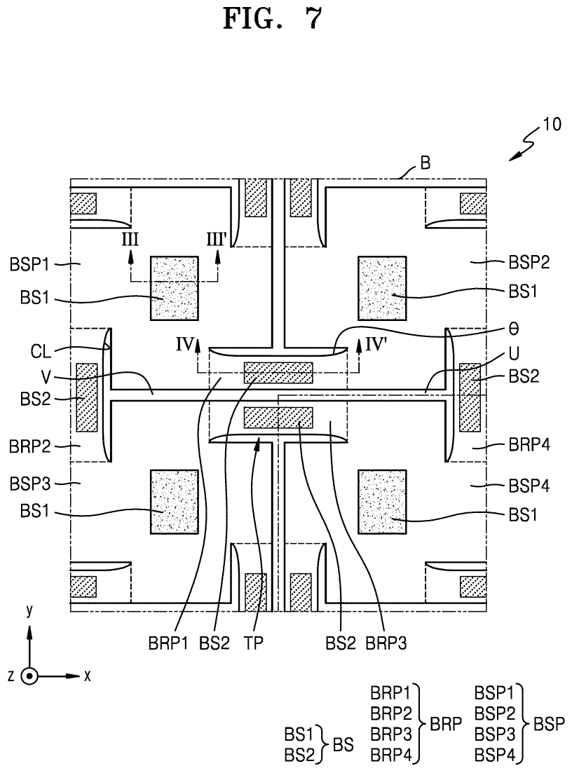

The flexible area FA may be a flexible area that has flexibility and thus is easily bendable, foldable, or stretchable. The flexible area FA has relatively low hardness compared to the hard area HA and thus may be easily stretched or contracted. In an embodiment, the flexible area FA may include a plurality of through-portions (FIGS. 4A and 7). The through-portions may pass through the display panel 10. The through-portions may be areas in which elements of the display panel 10 are not arranged. Accordingly, the display panel 10 may be easily stretched and/or contracted, and the display apparatus 1 as described above may be stretched or contracted in various directions. In an embodiment, for example, the display apparatus 1 is contractible in at least one of a first direction (e.g., one of an x-axis direction or y-axis direction) and a second direction perpendicular to the first direction. Through this behavior, the display apparatus 1 may have a three-dimensional shape. The flexible area FA as described above may include the display area DA and/or the sensor area SA.

The hard area HA may be a rigid area that has rigidity and thus is not easily bendable. In an embodiment, the hard area HA may be an area in which a through-hole is not arranged. Accordingly, the hard area HA may be an area that is not easily stretchable and/or contractable. A driving circuit DIC may be arranged in the hard area HA. The driving circuit DIC may be configured to generate signals and voltages for driving the display panel 10. The driving circuit DIC may be formed as an integrated circuit (“IC”) and attached onto the display panel 10 by using a chip-on-glass (“COG”) method, a chip-on-plastic (“COP”) method, or a chip-on-film (“COF”) method. However, in some embodiments, the display panel 10 may include only the flexible area FA, not the hard area HA.

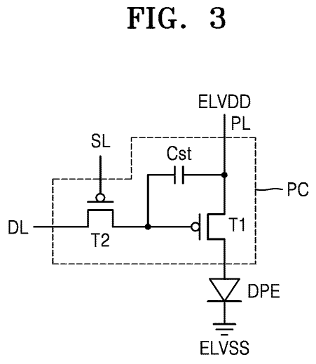

FIG. 3 is an equivalent circuit diagram of a pixel circuit PC applicable to a display panel according to an embodiment.

Referring to FIG. 3, the pixel circuit PC may be connected to a display element DPE. The pixel circuit PC may include a driving thin-film transistor T1, a switching thin-film transistor T2, and a storage capacitor Cst. Also, the display element DPE may emit red, green, or blue light or may emit red, green, blue, or white light.

The switching thin-film transistor T2 may be connected to a scan line SL and a data line DL and configured to transmit a data signal or a data voltage input through the data line DL, to the driving thin-film transistor T1 based on a scan signal or a switching voltage input through the scan line SL.

The storage capacitor Cst may be connected to the switching thin-film transistor T2 and a driving voltage line PL and configured to store a voltage corresponding to a difference between a voltage transmitted from the switching thin-film transistor T2 and a first power voltage ELVDD supplied through the driving voltage line PL.

The driving thin-film transistor T1 may be connected to the driving voltage line PL and the storage capacitor Cst and configured to control a driving current flowing from the driving voltage line PL to the display element DPE according to the voltage stored in the storage capacitor Cst. The display element DPE may emit light having a certain luminance according to the driving current. An opposite electrode of the display element DPE may be configured to receive a second power voltage ELVSS.

Though it is shown in FIG. 3 that the pixel circuit PC includes two thin-film transistors and one storage capacitor, the pixel circuit PC may include three or more thin-film transistors.

FIG. 4A is a schematic enlarged plan view of a region A of FIG. 2, and FIG. 4B is a plan view of the display panel 10 of FIG. 4A stretched in a first direction and a second direction.

Referring to FIG. 4A, the display area DA may include a display portion DP and a connection portion CP. The display portion DP may include a first display portion DP1 and a second display portion DP2. In an embodiment, the display area DA may include a plurality of first display portions DP1 and a plurality of second display portions DP2. The display area DA may include a plurality of connection portions CP. In an embodiment, for example, the connection portions CP may include a first connection portion CP1, a second connection portion CP2, a third connection portion CP3, and a fourth connection portion CP4.

The plurality of display portions DP may be spaced apart from each other in the first direction and/or the second direction. In an embodiment, the first direction and the second direction may be orthogonal to each other. In an embodiment, for example, the first direction may be an x-direction or −x direction of FIGS. 4A and 4B, and the second direction may be a y-direction or −y direction of FIGS. 4A and 4B. In another embodiment, the first direction and the second direction may form an acute angle or an obtuse angle with each other. In the following description, a case in which the first direction (e.g., x-direction or −x direction) and the second direction (e.g., y-direction or −y direction) are orthogonal to each other is mainly described in detail.

The first display portion DP1 and the second display portion DP2 may be spaced apart from each other in the first direction (e.g., x-direction or −x direction) and/or the second direction (e.g., y-direction or −y direction). In an embodiment, the plurality of display portions DP adjacent to each other may be spaced apart from each other by a first distance d1 in the first direction (e.g., x-direction or −x direction). In an embodiment, the plurality of display portions DP adjacent to each other may be spaced apart from each other by a second distance d2 in the second direction (e.g., y-direction or −y direction).

The first display portion DP1 may be connected to at least one connection portion CP. In an embodiment, for example, the first display portion DP1 may be connected to one connection portion CP. As another example, the first display portion DP1 may be connected to the plurality of connection portions CP.

The second display portion DP2 may be connected to at least one connection portion CP. In an embodiment, for example, the second display portion DP2 may be connected to one connection portion CP. As another example, the second display portion DP2 may be connected to the plurality of connection portions CP. The second display portion DP2 may be similar to the first display portion DP1.

The connection portion CP may extend between the display portions DP adjacent to each other. In an embodiment, each display portion DP may be connected to four connection portions CP. Four connection portions CP connected to one display portion DP may extend in different directions, and each connection portion CP may be connected to another display portion DP arranged adjacent to the aforementioned display portion DP.

In an embodiment, the connection portion CP may extend from the first display portion DP1 to the second display portion DP2. The first display portion DP1 and the second display portion DP2 may be connected to each other by the connection portion CP. In an embodiment, for example, the first connection portion CP1 may extend from the first display portion DP1 to the second display portion DP2. Accordingly, the first display portion DP1 and the second display portion DP2 may be connected to each other by the first connection portion CP1, and the first display portion DP1, the second display portion DP2, and the first connection portion CP1 may be integrally provided as a single body.

One of the plurality of connection portions CP may extend in a first direction (e.g., x-direction or −x direction). Another of the plurality of connection portions CP may extend in a second direction (e.g., y-direction or −y direction) crossing the first direction. In an embodiment, for example, the first connection portion CP1 and the third connection portion CP3 may extend in the first direction (e.g., x-direction or −x direction). The second connection portion CP2 and the fourth connection portion CP4 may extend in the second direction (e.g., y-direction or −y direction).

The display area DA is included in the flexible area FA (see FIG. 2A), and thus, a through-portion TP may be arranged in the display area DA. As described above with reference to FIG. 2, the through-portion TP may pass through the display panel 10. The through-portion TP may pass through an upper surface of the display panel 10 and a lower surface of the display panel 10. Accordingly, elements of the display panel 10 may not be arranged in the through-portion TP. The display panel 10 includes the through-portion TP, and thus, flexibility of the display area DA may be improved.

In an embodiment, at least a portion of he through-portion TP may be defined as an edge DPE1 of the first display portion DP1, an edge DPE2 of the second display portion DP2, and an edge CPE1 of the first connection portion CP1. In an embodiment, at least a portion of the through-portion TP may be defined as the edge DPE1 of the first display portion DP1, the edge DPE2 of the second display portion DP2, the edge CPE1 of the first connection portion CP1, and an edge CPE2 of the second connection portion CP2.

A portion of one display portion DP and the connection portions CP extending therefrom may be defined as a basic unit U. The basic unit U may be repeatedly arranged in the first direction (e.g., x-direction or −x direction) and the second direction (e.g., y-direction or −y direction), and it may be understood that the display area DA includes repeatedly arranged basic units U connected to each other. Two basic units U adjacent to each other may be symmetrical to each other. In an embodiment, for example, in FIG. 4A, two basic units U adjacent to each other in the left-right direction may be bilaterally symmetric with respect to an axis of symmetry positioned between the two basic units U and parallel to the second direction (e.g., y-direction or −y direction). Similarly, in FIG. 4A, two basic units U adjacent to each other in the vertical direction may be bilaterally symmetric with respect to an axis of symmetric positioned between the two basic units U and parallel to the first direction (e.g., x-direction or −x direction).

The basic units U adjacent to each other among the plurality of basic units U, e.g., the four basic units U shown in FIG. 4A, form a closed loop CL therebetween, and the closed loop CL may define a separated region V that is an empty space. The separated region V may be defined as the closed loop CL formed by edges of the plurality of display portions DP and edges of the plurality of connection portions CP. The separated region V may overlap the through-portion TP of the display panel 10.

In an embodiment, an angle θ between the edge CPE1 of the connection portion CP and the edge DPE2 of the second display portion DP2 may be an acute angle. When an external force pulling the display panel 10 is applied, as shown in FIG. 4B, an angle θ′ (θ′>0) between the edge CPE1 of the first connection portion CP1 and the edge DPE2 of the second display portion DP2 may increase, an area or shape of a separated region V′ may be changed, and a position of the display portion DP may also be changed.

When the aforementioned external force is applied, each display portion DP may be rotated at a certain angle by the aforementioned change in the angle θ′, increase in the area of the separated region V′, and/or change in the shape of the separated region V′. Distances between the display portions DP, e.g., a first distance d1′ and a second distance d2′, may be different for each position due to rotation of each of the display portions DP. That is, a distance between the display portions DP adjacent to each other among the plurality of display portions DP may be changed by an external force, and the plurality of connection portions CP may be stretched by an external force.

When the external force pulling the display panel 10 is applied, stress may be concentrated on the edge CPE1 of the first connection portion CP1 and the edge DPE2 of the second display portion DP2. In this case, in order to prevent damage to the display panel 10, the closed loop CL defining the separated region V may include a curve.

A first pixel PX1 and a second pixel PX2 may be arranged in the first display portion DP1 and the second display portion DP2, respectively. Each of the first pixel PX1 and the second pixel PX2 may include a red subpixel Pr, a green subpixel Pg, and a blue subpixel Pb. The red subpixel Pr, the green subpixel Pg, and the blue subpixel Pb may emit red light, green light, and blue light, respectively. In another embodiment, each of the first pixel PX1 and the second pixel PX2 may include a red subpixel Pr, a green subpixel Pg, a blue subpixel Pb, and a white subpixel. The red subpixel Pr, the green subpixel Pg, the blue subpixel Pb, and the white subpixel may emit red light, green light, blue light, and white light, respectively. In the following description, a case in which each of the first pixel PX1 and the second pixel PX2 includes the red subpixel Pr, the green subpixel Pg, and the blue subpixel Pb is mainly described in detail.

An arrangement structure of subpixels of the first pixel PX1 and an arrangement structure of subpixels of the second pixel PX2 may include a stripe structure. In an embodiment, for example, the red subpixel Pr, the green subpixel Pg, and the blue subpixel Pb may be arranged parallel to each other in the first direction (e.g., x-direction or −x direction) or the second direction (e.g., y-direction or −y direction). In another embodiment, the arrangement structure of subpixels of the first pixel PX1 and the arrangement structure of subpixels of the second pixel PX2 may include an S-stripe structure. Also, in another embodiment, the arrangement structure of subpixels of the first pixel PX1 and the arrangement structure of subpixels of the second pixel PX2 may include a Pentile structure.

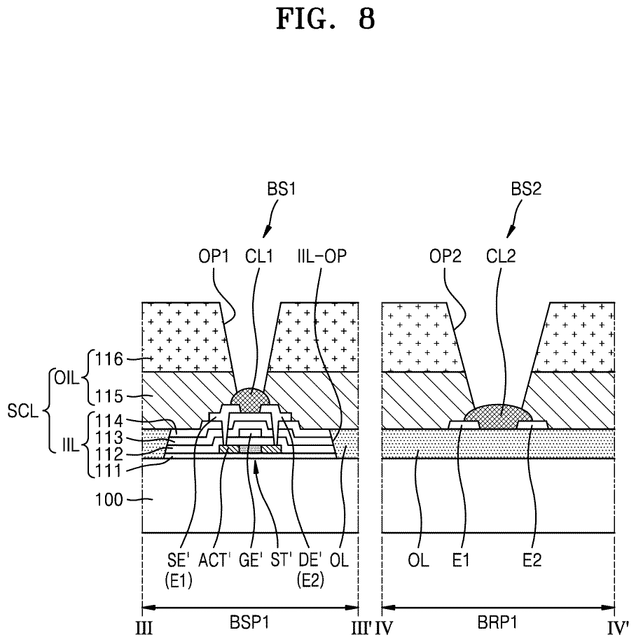

In an embodiment, the display panel 10 as described above may be applied to the sensor area SA of FIG. 2. In an embodiment, the display panel 10 as described above may be applied to the sensor area SA and the display area DA of FIG. 2. The sensor area SA will be described in detail with reference to FIGS. 7 and 8.

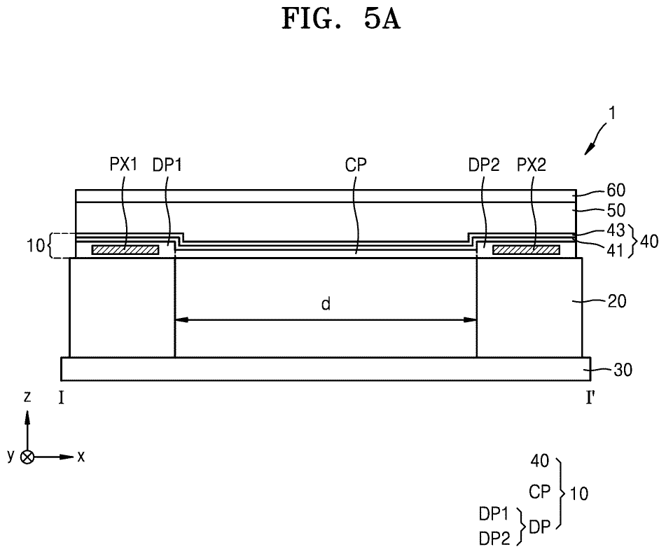

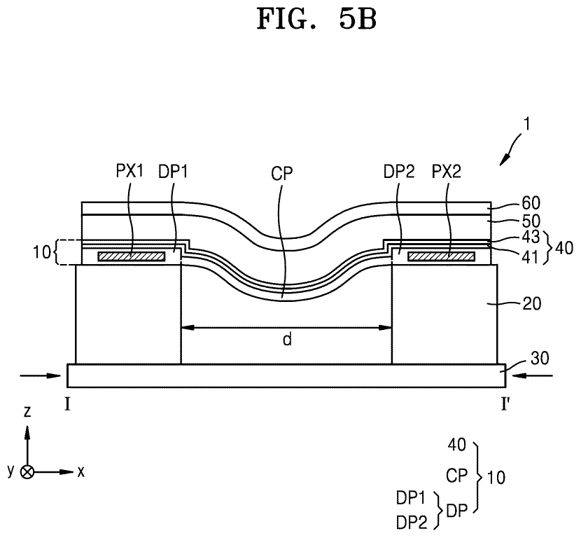

FIGS. 5A and 5B are schematic cross-sectional views of the display apparatus 1 according to an embodiment. In detail, FIGS. 5A and 5B are diagrams of a cross-section (of the display panel 10 taken along line I-I′ of FIG. 4A. FIG. 5 illustrates the display apparatus 1 before an external force is applied to the display apparatus 1, and FIG. 5B illustrates the display apparatus 1 after an external force is applied to the display apparatus 1.

Referring to FIGS. 5A and 5B, the display apparatus 1 may include the display panel 10, a pillar layer 20, a flexible substrate 30, an optical functional layer 50, and a cover window 60.

The display panel 10 may include the display portion DP, the connection portion CP, and an encapsulation layer 40, and the display portion DP may include the first display portion DP1 and the second display portion DP2. Also, the first pixel PX1 may be arranged in the first display portion DP1, and the second pixel PX2 may be arranged in the second display portion DP2. The first display portion DP1 and the second display portion DP2 may be portions in which images are displayed through the first pixel PX1 and the second pixel PX2, respectively. The connection portion CP may connect the first display portion DP1 and the second display portion DP2 to each other. The first display portion DP1 and the second display portion DP2 may be connected to each other through the connection portion CP.

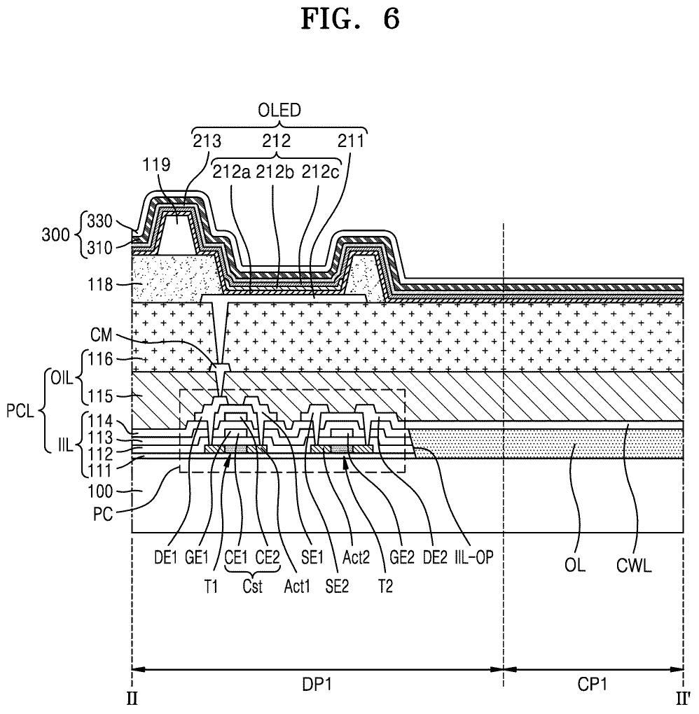

In addition, as shown in FIG. 6 to be described below, the display portion DP of the display panel 10 may include insulating layers. Some insulating layers included in the first display portion DP1 may be omitted from the connection portion CP. Accordingly, because some insulating layers may be omitted from the connection portion CP, a thickness of the connection portion CP may be less than a thickness of the display portion DP.

The pillar layer 20 may be arranged under the display panel 10. The pillar layer 20 may support the display panel 10. Although an external force is applied to the display apparatus 1, a shape of the pillar layer 20 may not be changed.

In an embodiment, the pillar layer 20 may be arranged under the display portion DP of the display panel 10. That is, the pillar layer 20 may overlap the display portion DP and not overlap the connection portion CP. A plurality of pillar layers 20 may be spaced apart from each other. The plurality of pillar layers 20 may be arranged under the plurality of display portions DP, respectively, where the display portions DP are spaced apart from each other.

The flexible substrate 30 may be arranged under the pillar layer 20. The flexible substrate 30 may overlap the display portion DP and the connection portion CP. The flexible substrate 30 may include a flexible material. The flexible substrate 30 may be more flexible than the pillar layer 20.

The encapsulation layer 40 may be disposed on the display portion DP and the connection portion CP. The encapsulation layer 40 may cover the first and second pixels PX1 and PX2. In an embodiment, the encapsulation layer 40 may include at least one inorganic layer. The at least one inorganic layer may include one or more inorganic insulating materials, such as silicon oxide (SiOₓ), silicon nitride (SiNₓ), silicon oxynitride (SiOₓN_{y}), aluminum oxide (Al₂O₃), titanium oxide (TiO₂), tantalum oxide (Ta₂O₅), hafnium oxide (HfO₂), or zinc oxide (ZnOₓ), and may be formed by chemical vapor deposition (“CVD”) or the like. The ZnOₓ may be ZnO and/or ZnO₂.

In an embodiment, as shown in FIGS. 5A and 5B, the encapsulation layer 40 may be disposed on the display portion DP and the connection portion CP. The encapsulation layer 40 may include a first inorganic layer 41 and a second inorganic layer 43. Alternatively, though not shown, the encapsulation layer 40 may include an organic layer between the first inorganic layer 41 and the second inorganic layer 43. When the encapsulation layer 40 includes the organic layer, the organic layer may be disposed only on the display portion DP and not on the connection portion CP. That is, only the first inorganic layer 41 and the second inorganic layer 43 may be arranged over the connection portion CP.

The optical functional layer 50 may be disposed on the encapsulation layer 40. The optical functional layer 50 may include an anti-reflection layer. The anti-reflection layer may reduce reflectance of light (external light) incident from the outside toward the display apparatus 1. In an embodiment, the optical functional layer 50 may include a polarization film. Alternatively, the optical functional layer 50 may include a filter plate including a black matrix and color filters.

Though not shown in FIGS. 5A and 5B, a touch screen layer may be between the encapsulation layer 40 and the optical functional layer 50. The touch screen layer may obtain coordinate information corresponding to an external input, e.g., a touch event. The touch screen layer may include a touch electrode, and touch lines connected to the touch electrode. The touch screen layer may sense an external input based on a magnetic capacitance method or a mutual capacitance method.

The cover window 60 may be disposed on the optical functional layer 50. The cover window 60 may protect the display panel 10.

In addition, as shown in FIG. 5B, when an external force is applied to the display apparatus 1 (e.g., when an external force is applied to the flexible substrate 30), shapes and/or positions of some elements of the display apparatus 1 may be changed. In an embodiment, for example, when pressure is applied to the flexible substrate 30, a distance between the display portions DP of the display panel 10 and a distance d between the pillar layers 20 may decrease. Also, the connection portion CP of the display panel 10 may be bent. At least a portion of the encapsulation layer 40, at least a portion of the optical functional layer 50, and/or at least a portion of the cover window 60 may be bent. Accordingly, when the external force is applied to the display apparatus 1, the distance between the display portions DP of the display panel 10 the distance d between the pillar layers 20 may be changed, and a shape of each of the display portions DP of the display panel 10 and the pillar layers 20 may not be changed. Therefore, because the shape of each of the display portions DP and the pillar layers 20 does not change, the first and second pixels PX1 and PX2 arranged in the display portion DP may be protected. The display apparatus 1 may be changed into various forms while protecting the first and second pixels PX1 and PX2.

FIG. 6 is a schematic cross-sectional view of the display panel 10 taken along line II-II′ of FIG. 4A. That is, FIG. 6 is a schematic cross-sectional view of a portion of the display area DA in the display apparatus, according to an embodiment.

Referring to FIG. 6, the display panel 10 may include a substrate 100, a pixel circuit layer PCL, an organic light-emitting diode OLED, and an encapsulation layer 300.

In an embodiment, the substrate 100 may include at least one base layer and at least one barrier layer. In an embodiment, the barrier layer may be arranged over the base layer. In another embodiment, the substrate 100 may include glass.

The base layer may include a polymer resin, such as polyethersulfone, polyarylate, polyetherimide, polyethylene naphthalate, polyethylene terephthalate, polyphenylene sulfide, polyimide, polycarbonate, cellulose triacetate, and cellulose acetate propionate.

The barrier layer is a barrier layer preventing penetration of an external foreign material and may include a single layer or multi-layer including an inorganic material, such as SiNₓ, silicon oxide (SiO₂), and/or silicon oxynitride (SiON).

The pixel circuit layer PCL may be arranged in the first display portion DP1. The pixel circuit layer PCL may include a pixel circuit. In an embodiment, the pixel circuit layer PCL may include a plurality of pixel circuits. The pixel circuit layer PCL may include the driving thin-film transistor T1, the switching thin-film transistor T2, and the storage capacitor Cst. Although a stacked structure on the first display portion DP1 is described with reference to FIG. 6, this may equally be applied to the second display portion DP2 (see FIG. 4A).

The pixel circuit layer PCL may include an inorganic insulating layer IIL and an organic insulating layer OIL arranged under and/or over elements of the driving thin-film transistor T1. The inorganic insulating layer IIL may include a buffer layer 111, a first gate insulating layer 112, a second gate insulating layer 113, and an interlayer-insulating layer 114. The organic insulating layer OIL may include a first organic insulating layer 115 and a second organic insulating layer 116. The driving thin-film transistor T1 may include a first semiconductor layer Act1, a first gate electrode GE1, a first source electrode SE1, and a first drain electrode DE1.

The buffer layer 111 may be disposed on the substrate 100. The buffer layer 111 may include an inorganic insulating material, such as SiN_{X}, SiON, or SiO₂, and may include a single layer or multi-layer including aforementioned the inorganic insulating material.

The first semiconductor layer Act1 may be disposed on the buffer layer 111. The first semiconductor layer Act1 may include polysilicon. Alternatively, the first semiconductor layer Act1 may include amorphous silicon, an oxide semiconductor, or an organic semiconductor. The first semiconductor layer Act1 may include a channel region, and a drain region and a source region, which are arranged on opposite sides of the channel region, respectively.

The first gate electrode GE1 may overlap the channel region. The first gate electrode GE1 may include a low-resistance metal material. The first gate electrode GE1 may include a conductive material including molybdenum (Mo), aluminum (Al), copper (Cu), or titanium (Ti), and may include a multi-layer or single layer including the above conductive material. The first gate electrode GE1 may be integrally formed as a single body with a lower electrode CE1 of the storage capacitor Cst.

The first gate insulating layer 112 between the first semiconductor layer Act1 and the first gate electrode GE1 may include an inorganic insulating material, such as SiO₂, SiNₓ, SiON, Al₂O₃, TiO₂, Ta₂O₅, HfO₂, and/or ZnO.

The second gate insulating layer 113 may cover the first gate electrode GE1. Similar to the first gate insulating layer 112, the second gate insulating layer 113 may include an inorganic insulating material, such as SiO₂, SiNₓ, SiON, Al₂O₃, TiO₂, Ta₂O₅, HfO₂, and/or ZnO.

An upper electrode CE2 of the storage capacitor Cst may be disposed on the second gate insulating layer 113. The upper electrode CE2 may overlap the first gate electrode GE1 thereunder. In this case, the upper electrode CE2 and the first gate electrode GE1 of the driving thin-film transistor T1, which overlap with the second gate insulating layer 113 therebetween, may form the storage capacitor Cst. That is, the first gate electrode GE1 of the driving thin-film transistor T1 may function as the lower electrode CE1 of the storage capacitor Cst.

As described above, the storage capacitor Cst may overlap the driving thin-film transistor T1. In some embodiments, the storage capacitor Cst may also not overlap the driving thin-film transistor T1.

The upper electrode CE2 may include Al, platinum (Pt), palladium (Pd), silver (Ag), magnesium (Mg), gold (Au), nickel (Ni), neodymium (Nd), iridium (Ir), chromium (Cr), calcium (Ca), Mo, Ti, tungsten (W), and/or Cu, and may include a single layer or multi-layer including the aforementioned material.

An interlayer-insulating layer 114 may cover the upper electrode CE2. The interlayer-insulating layer 114 may include SiO₂, SiN_{X}, SiON, Al₂O₃, TiO₂, Ta₂O₅, HfO₂, or ZnO. The interlayer-insulating layer 114 may include a single layer or multi-layer including the aforementioned inorganic insulating material.

The first drain electrode DE1 and the first source electrode SE1 may each be positioned on the interlayer-insulating layer 114. The first drain electrode DE1 and the first source electrode SE1 may each include a material having excellent conductivity. The first drain electrode DE1 and the first source electrode SE1 may each include a conductive material including Mo, Al, Cu, or Ti, and may include a multi-layer or single layer including the above conductive material. In an embodiment, the first drain electrode DE1 and the first source electrode SE1 may each have a multi-layered structure of Ti/Al/Ti.

The switching thin-film transistor T2 may include a second semiconductor layer Act2, a second gate electrode GE2, a second drain electrode DE2, and a second source electrode SE2. The second semiconductor layer Act2, the second gate electrode GE2, the second drain electrode DE2, and the second source electrode SE2 are similar to the first semiconductor layer Act1, the first gate electrode GE1, the first drain electrode DE1, and the first source electrode SE1, respectively, and thus, detailed descriptions thereof are omitted.

The first organic insulating layer 115 may cover the first drain electrode DE1 and the first source electrode SE1. The first organic insulating layer 115 may include an organic material. In an embodiment, for example, the first organic insulating layer 115 may include an organic insulating material including a general-purpose polymer, such as poly(methyl methacrylate) (“PMMA”) or polystyrene (“PS”), a polymer derivate having a phenol-based group, an acrylic polymer, an imide-based polymer, an aryl ether-based polymer, an amide-based polymer, a fluorine-based polymer, a p-xylene-based polymer, a vinyl alcohol-based polymer, and any blends thereof.

The second organic insulating layer 116 may include an organic material. The second organic insulating layer 116 may include an organic insulating material including a general-purpose polymer, such as PMMA or PS, a polymer derivate having a phenol-based group, an acrylic polymer, an imide-based polymer, an aryl ether-based polymer, an amide-based polymer, a fluorine-based polymer, a p-xylene-based polymer, a vinyl alcohol-based polymer, or any blends thereof.

In an embodiment, a passivation layer (not shown) may be additionally arranged over the second organic insulating layer 116. That is, the passivation layer may be between the organic light-emitting diode OLED and the second organic insulating layer 116. The passivation layer may include a single-layer or multi-layer including an inorganic material, such as SiNₓ and/or SiO₂.

The organic light-emitting diode OLED may be disposed on the pixel circuit layer PCL. The organic light-emitting diode OLED may be arranged in each of the plurality of display portions DP. The organic light-emitting diode OLED may include a pixel electrode 211, an intermediate layer 212, and an opposite electrode 213. The pixel electrode 211 may be arranged over the second organic insulating layer 116 or the passivation layer. The pixel electrode 211 may be electrically connected to a contact metal CM through a contact hole of the second organic insulating layer 116. Accordingly, the organic light-emitting diode OLED may be electrically connected to the pixel circuit PC.

The pixel electrode 211 may include a conductive oxide, such as indium tin oxide (“ITO”), indium zinc oxide (“IZO”), zinc oxide (ZnO), indium oxide (In₂O₃), indium gallium oxide (“IGO”), or aluminum zinc oxide (“AZO”). In another embodiment, the pixel electrode 211 may include a reflective layer including Ag, Mg, Al, Pt, Pd, Au, Ni, Nd, Ir, Cr, or any compounds thereof. In another embodiment, the pixel electrode 211 may further include a layer including ITO, IZO, ZnO, or In₂O₃ over/under the reflective layer.

A bank layer 118 defining an opening therein, which exposes a central portion of the pixel electrode 211 may be disposed on the pixel electrode 211. The bank layer 118 may include an organic insulating material and/or an inorganic insulating material. An opening of the bank layer 118 may define an emission area of the organic light-emitting diode OLED. In an embodiment, for example, a width of the opening of the bank layer 118 may correspond to a width of the emission area.

A spacer 119 may be disposed on the bank layer 118. A mask sheet may be used in a method of manufacturing the display panel 10, in which case the mask sheet may enter the opening of the bank layer 118 or adhere to the bank layer 118. The spacer 119 may prevent a portion of the substrate 100 and the multi-layer on the substrate 100 from being damaged or broken by the mask sheet when a deposition material is deposited on the substrate 100.

The spacer 119 may include an organic material, such as polyimide. Alternatively, the spacer 119 may include an inorganic insulating material, such as SiNₓ or SiO₂, or may include an organic insulating material and an inorganic insulating material.

In an embodiment, the spacer 119 may include a material different from a material of the bank layer 118. Also, in another embodiment, the spacer 119 may include the same material as the bank layer 118, in which case the bank layer 118 and the spacer 119 may be formed together in a mask process using a half-tone mask.

The intermediate layer 212 may be disposed on the bank layer 118. The intermediate layer 212 may include an emission layer 212b arranged in the opening of the bank layer 118. The emission layer 212b may include a polymer organic material or a low-molecular weight organic material emitting light of a certain color.

A first functional layer 212a and a second functional layer 212c may be arranged under and over the emission layer 212b. The first functional layer 212a may include, e.g., a hole transport layer (“HTL”), or a HTL and a hole injection layer (“HIL”). The second functional layer 212c is an element disposed on the emission layer 212b and may be provided optionally. The second functional layer 212c may include an electron transport layer (“ETL”) and/or an electron injection layer (“EIL”). Similar to the opposite electrode 213 described below, the first functional layer 212a and/or the second functional layer 212c may be common layers formed to entirely cover the substrate 100.

The opposite electrode 213 may include a conductive material having a low work function. In an embodiment, for example, the opposite electrode 213 may include a (semi-)transparent layer including Ag, Mg, Al, Pt, Pd, Au, Ni, Nd, Ir, Cr, lithium (Li), Ca, or any alloys thereof. Alternatively, the opposite electrode 213 may further include a layer including ITO, IZO, ZnO, or In₂O₃ over the (semi-)transparent layer including the aforementioned material.

In some embodiments, a capping layer (not shown) may be further arranged over the opposite electrode 213. The capping layer may include lithium fluoride (LiF), an inorganic material, and/or an organic material.

The encapsulation layer 300 may be disposed on the opposite electrode 213. In an embodiment, the encapsulation layer 300 may include at least one inorganic encapsulation layer. In another embodiment, the encapsulation layer 300 may include at least one inorganic encapsulation layer and at least one organic encapsulation layer. In an embodiment, FIG. 6 illustrates that the encapsulation layer 300 includes a first inorganic encapsulation layer 310 and a second inorganic encapsulation layer 330, which are sequentially stacked. In FIG. 6, the second inorganic encapsulation layer 330 is directly disposed on the first inorganic encapsulation layer 310, and accordingly, the first inorganic encapsulation layer 310 and the second inorganic encapsulation layer 330 may be in direct contact with each other on the organic light-emitting diode OLED.

The first inorganic encapsulation layer 310 and the second inorganic encapsulation layer 330 may each include at least one inorganic material from among Al₂O₃, TiO₂, Ta₂O₅, HfO₂, ZnO, SiO₂, SiNₓ, and SiON.

Though not shown, a touch electrode layer may be arranged over the encapsulation layer 300, and an optical functional layer may be arranged over the touch electrode layer. The touch electrode layer may obtain coordinate information corresponding to an external input, e.g., a touch event. The optical functional layer may reduce the reflectance of light (external light) incident from the outside towards the display apparatus, and/or enhance color purity of light emitted from the display apparatus. In an embodiment, the optical functional layer may include a retarder and/or a polarizer. The retarder may include a film-type retarder or a liquid crystal coating-type retarder, and may include a λ/2 retarder and/or a λ/4 retarder. The polarizer may also include a film-type polarizer or a liquid crystal coating-type polarizer. The film-type polarizer may include a stretchable synthetic resin film, and the liquid crystal coating-type polarizer may include liquid crystals arranged in a certain arrangement. Each of the retarder and the polarizer may further include a protective film.

In another embodiment, the optical functional layer may include a black matrix and color filters. The color filters may be arranged by taking into account colors of light emitted from the pixels of the display panel, respectively. Each of the color filters may include red, green, or blue pigment or dye. Alternatively, each of the color filters may further include quantum dots in addition to the aforementioned pigment or dye. Alternatively, some of the color filters may not include the aforementioned pigment or dye, and may include scattering particles, such as titanium oxide.

In another embodiment, the optical functional layer may include a destructive interference structure. The destructive interference structure may include a first reflective layer and a second reflective layer, which are disposed on different layers. First reflected light and second reflected light reflected from the first reflective layer and the second reflective layer, respectively, may destructively interfere with each other, and accordingly, the reflectance of external light may be reduced.

An adhesive member may be between the touch electrode layer and the optical functional layer. For the adhesive member, a general adhesive member known in the art may be employed without limitation. The adhesive member may include a pressure sensitive adhesive (“PSA”).

In addition, the inorganic insulating layer IIL arranged in the first display portion DP1 may not be arranged in the connection portion CP. That is, the inorganic insulating layer IIL may be removed from the connection portion CP. The connection portion CP of the display panel 10 may be bent as described with reference to FIGS. 4A and 4B, and thus, it is important for the connection portion CP to have flexibility. The inorganic insulating layer IIL has less flexibility than the organic insulating layer OIL and is particularly vulnerable to cracking. The cracking may provide a path through which moisture and oxygen penetrate into the first display portion DP1 or the like. Accordingly, as shown in FIG. 6, as the inorganic insulating layer IIL is removed from the connection portion CP, the flexibility of the connection portion CP may be improved, and an occurrence of cracking may be significantly reduced.

The inorganic insulating layer IIL may defined an open portion IIL-OP therein to correspond to the connection portion CP. The open portion IIL-OP may refer to a region from which the inorganic insulating layer IIL is removed. An upper surface of the substrate 100 may be exposed through the open portion IIL-OP. Although the region from which the inorganic insulating layer IIL is removed is defined as the open portion IIL-OP, the inorganic insulating layer IIL remaining on a plane may have an island shape corresponding to the first display portion DP1. The open portion IIL-OP may extend from the connection portion CP toward the first display portion DP1. That is, an area of the open portion IIL-OP may be greater than or equal to an area of the connection portion CP.

In an embodiment, an organic material layer OL may be arranged in the open portion IIL-OP of the inorganic insulating layer IIL. The organic material layer OL may be buried in the open portion IIL-OP of the inorganic insulating layer IIL and fill a step of the inorganic insulating layer IIL, which is formed by the open portion IIL-OP. A thickness of the organic material layer OL may be substantially equal to a height of the step of the inorganic insulating layer IIL, which is formed by the open portion IIL-OP. By filling the open portion IIL-OP of the inorganic insulating layer IIL with the organic material layer OL, the flexibility of the connection portion CP may be improved, and by planarizing a region in which a connection wire CWL to be described below as much as possible, reliability of a connection wire CWL may be improved.

The connection wire CWL may be disposed on the organic material layer OL. In this case, the connection wire CWL may be electrically connected to the pixel circuit PC. The pixel circuit PC of the first display portion DP1 may be electrically connected to a pixel circuit of another adjacent display portion (e.g., a pixel circuit of the second display portion DP2) through the connection wire CWL. The connection wire CWL may include a material having excellent conductivity. The connection wire CWL may include a conductive material including Mo, Al, Cu, or Ti, and may include a multi-layer or single layer including the above conductive material. In an embodiment, the connection wire CWL may have a multi-layered structure of Ti/Al/Ti. The connection wire CWL may refer to a wire electrically connecting display portions to each other, and may include, e.g., wires through which various signals and power are transmitted, such as a data line, a scan line, and a power supply line.

FIG. 7 is a schematic enlarged plan view of a region B of FIG. 2, and FIG. 8 is a schematic cross-sectional view of the display panel 10 taken along lines III-III′ and IV-IV″. That is, FIGS. 7 and 8 are a schematic plan view and a schematic cross-sectional view, respectively, of a portion of the sensor area SA in the display apparatus, according to an embodiment.

Referring to FIG. 7, the sensor area SA may include a base portion BSP and a bridge portion BRP. The base portion BSP may include a first base portion BSP1 and a second base portion BSP2. In an embodiment, the sensor area SA may include a plurality of first base portions BSP1 and a plurality of second base portions BSP2. The sensor area SA may include a plurality of bridge portions BRP. In an embodiment, for example, the bridge portion BRP may include a first bridge portion BRP1, a second bridge portion BRP2, a third bridge portion BRP3, and a fourth bridge portion BRP4.

The plurality of base portions BSP may be spaced apart from each other in a first direction and/or a second direction. In an embodiment, the first direction and the second direction may be orthogonal to each other. In an embodiment, for example, the first direction may be an x-direction or −x direction of FIG. 7, and the second direction may be a y-direction or −y direction. In another embodiment, the first direction and the second direction may form an acute angle or an obtuse angle with each other. Hereinafter, similar to FIGS. 4A and 4B, a case in which the first direction (e.g., x-direction or −x direction) and the second direction (e.g., y-direction or −y direction) are orthogonal to each other is mainly described in detail. The first base portion BSP1 and the second base portion BSP2 may be spaced apart from each other in the first direction (e.g., x-direction or −x direction) and/or the second direction (e.g., y-direction or −y direction).

The first base portion BSP1 may be connected to at least one bridge portion BRP. In an embodiment, for example, the first base portion BSP1 may be connected to one bridge portion BRP. As another example, the first base portion BSP1 may be connected to the plurality of bridge portions BRP.

The second base portion BSP2 may be connected to at least one bridge portion BRP. In an embodiment, for example, the second base portion BSP2 may be connected to one bridge portion BRP. As another example, the second base portion BSP2 may be connected to the plurality of bridge portions BRP. The second base portion BSP2 may be similar to the first base portion BSP1.

The bridge portion BRP may extend between adjacent base portions BSP. In an embodiment, each base portion BSP may be connected to four bridge portions BRP. Four bridge portions BRP connected to one base portion BSP may extend in different directions, and each bridge portion BRP may be connected to another base portion BSP arranged adjacent to the aforementioned one base portion BSP.

In an embodiment, the bridge portion BRP may extend from the first base portion BSP1 to the second base portion BSP2. The first base portion BSP1 and the second base portion BSP2 may be connected to each other by the bridge portion BRP. In an embodiment, for example, the first bridge portion BRP1 may extend from the first base portion BSP1 to the second base portion BSP2. Accordingly, the first base portion BSP1 and the second base portion BSP2 may be connected to each other by the first bridge portion BRP1, and the first base portion BSP1, the second base portion BSP2, and the first bridge portion BRP1 may be integrally provided as a single body.

One of the plurality of bridge portions BRP may extend in the first direction (e.g., x-direction or −x direction). Another of the plurality of bridge portions BRP may extend in the second direction (e.g., y-direction or −y direction) crossing the first direction. In an embodiment, for example, the first bridge portion BRP1 and the third bridge portion BRP3 may extend in the first direction (e.g., x-direction or −x direction). The second bridge portion BRP2 and the fourth bridge portion BRP4 may extend in the second direction (e.g., y-direction or −y direction).

The sensor area SA is included in the flexible area FA (see FIG. 2), similar to the display area DA, and thus, the through-portion TP may also be arranged in the sensor area SA. As described above with reference to FIG. 2, the through-portion TP may pass through the display panel 10. The through-portion TP may pass through the upper surface of the display panel 10 and the lower surface of the display panel 10. Accordingly, the elements of the display panel 10 may not be arranged in the through-portion TP. The display panel 10 includes the through-portion TP, and thus, flexibility of the sensor area SA may be effectively improved.

A portion of one base portion BSP and the bridge portions BRP extending therefrom may be defined as a base unit U. The basic unit U may be repeatedly arranged in the first direction (e.g., x-direction or −x direction) and the second direction (e.g., y-direction or −y direction), and it may be understood that the sensor area SA includes repeatedly arranged basic units U connected to each other. Two basic units U adjacent to each other may be symmetrical to each other. In an embodiment, for example, in FIG. 7, two basic units U adjacent to each other in the left-right direction may be bilaterally symmetric with respect to the axis of symmetry positioned between the two basic units U and parallel to the second direction (e.g., y-direction or −y direction). Similarly, in FIG. 7, two basic units U adjacent to each other in the vertical direction may be bilaterally symmetric with respect to the axis of symmetric positioned between the two basic units U and parallel to the first direction (e.g., x-direction or −x direction).

The basic units U adjacent to each other among the plurality of basic units U, e.g., the four basic units U shown in FIG. 7, form a closed loop CL therebetween, and the closed loop CL may define a separated region V that is an empty space. The separated region V may be defined as the closed loop CL formed by edges of the plurality of base portions BSP and edges of the plurality of bridge portions BRP. The separated region V may overlap the through-portion TP of the display panel 10.

In an embodiment, an angle θ between an edge of the bridge portion BRP and an edge of the second base portion BSP2 may be an acute angle. When an external force pulling the display panel 10 is applied, as in FIG. 4B, the angle θ between the edge of the first bridge portion BRP1 and the edge of the second base portion BSP2 may also increase in the sensor area SA, an area or shape of the separated region V may be changed, and a position of the base portion BSP may also be changed.

When the aforementioned external force is applied, each base portion BSP may be rotated at a certain angle by the aforementioned change in the angle θ, increase in the area of the separated region V, and/or change in the shape of the separated region V. A distance between the base portions BSP may be different for each position due to rotation of each base portion BSP. That is, a distance between the base portions BSP arranged adjacent to each other among the plurality of base portions BSP may be changed by the external force, and the plurality of bridge portions BRP may be stretched by the external force.

The sensor area SA may include a plurality of biosensors BS. The plurality of biosensors BS may include a plurality of first biosensors BS1 and a plurality of second biosensors BS2. The plurality of first biosensors BS1 may be arranged in the plurality of base portions BSP, respectively, and the plurality of second biosensors BS2 may be arranged in the plurality of bridge portions BRP, respectively. In an embodiment, for example, each of the first base portion BSP1 and the second base portion BSP2 may include a first biosensor BS1. Each of the first bridge portion BRP1, the second bridge portion BRP2, the third bridge portion BRP3, and the fourth bridge portion BRP4 may include a second biosensor BS2.

The plurality of biosensors BS may be electrical sensors of which electrical resistance changes when exposed to a target material. In an embodiment, the plurality of biosensors BS may each include an M13 bacteriophage. Accordingly, when M13 bacteriophages of the plurality of biosensors BS are exposed to the target material, a bundle arrangement of the M13 bacteriophages may change. In detail, when the plurality of biosensors BS are exposed to the target material, surface proteins of the M13 bacteriophages swell, such that an electron movement path may change, and current values or resistance values thereof may change.

Also, the plurality of biosensors BS may include M13 bacteriophages genetically modified differently from each other, respectively. In detail, the plurality of biosensors BS arranged in the plurality of base portions BSP and the plurality of bridge portions BRP may include peptides having different sequences, respectively. Accordingly, even when exposed to the same target material, each of the plurality of biosensors BS may detect different current values or resistance values. The plurality of biosensors BS will be described below in detail with reference to FIGS. 11A to 11E.

Referring to FIG. 8, the substrate 100, a sensor circuit layer SCL, and the plurality of biosensors BS may be arranged in the sensor area SA. Because the display area DA and the sensor area SA are arranged in one display panel 10, the substrate 100 may include the same material as the substrate 100 of FIG. 6.

The sensor circuit layer SCL may be arranged in the first base portion BSP1. The sensor circuit layer SCL is electrically connected to the plurality of biosensors BS and thus may sense the target material. The sensor circuit layer SCL may include a sensor circuit. In an embodiment, the sensor circuit layer SCL may include a sensor thin-film transistor ST′ of which resistance is variable when exposed to the target material. The sensor thin-film transistor ST′ may include a sensor semiconductor layer Act′, a sensor gate electrode GE′, a sensor source electrode SE′, and a sensor drain electrode DE′.

Also in the sensor area SA, the buffer layer 111 may be disposed on the substrate 100. The buffer layer 111 may include an inorganic insulating material, such as SiN_{X}, SiON, or SiO₂, and may include a single layer or multi-layer including aforementioned the inorganic insulating material.

The sensor semiconductor layer Act′ may be disposed on the buffer layer 111. The sensor semiconductor layer Act′ may include polysilicon. Alternatively, the sensor semiconductor layer Act′ may include amorphous silicon, an oxide semiconductor, or an organic semiconductor. The sensor semiconductor layer Act′ may include a channel region, and a drain region and a source region, which are arranged on opposite sides of the channel region, respectively.

The sensor gate electrode GE′ may overlap the channel region. The sensor gate electrode GE′ may include a low-resistance metal material. The sensor gate electrode GE′ may include a conductive material including Mo, Al, Cu, or Ti, and may include a multi-layer or single layer including the above conductive material.

The first gate insulating layer 112 between the sensor semiconductor layer Act′ and the sensor gate electrode GE′ may include an inorganic insulating material, such as SiO₂, SiN_{X}, SiON, Al₂O₃, TiO₂, Ta₂O₅, HfO₂, and/or ZnO.

The second gate insulating layer 113 may cover the sensor gate electrode GE′. Similar to the first gate insulating layer 112, the second gate insulating layer 113 may include an inorganic insulating material, such as SiO₂, SiN_{X}, SiON, Al₂O₃, TiO₂, Ta₂O₅, HfO₂, and/or ZnO.

The sensor source electrode SE′ and the sensor drain electrode DE′ may be arranged on opposite ends of the sensor semiconductor layer Act′, respectively. In an embodiment, the sensor source electrode SE′ may at least partially overlap the source region of the sensor semiconductor layer Act′. The sensor drain electrode DE′ may at least partially overlap the drain region of the sensor semiconductor layer Act′.

In an embodiment, the sensor source electrode SE′ and the sensor drain electrode DE′ may each include a material having excellent conductivity. At least one of the sensor source electrode SE′ and the sensor drain electrode DE′ may include a conductive material including Mo, Al, Cu, or Ti, and may include a multi-layer or single layer including the above conductive material. In an embodiment, at least one of the sensor source electrode SE′ and the sensor drain electrode DE′ may have a multi-layered structure of Ti/Al/Ti.

Similar to the display area DA, the organic insulating layer OIL may also be arranged in the sensor area SA. The organic insulating layer OIL may include the first organic insulating layer 115 and the second organic insulating layer 116. The first organic insulating layer 115 may cover the sensor drain electrode DE′ and the sensor source electrode SE′. The first organic insulating layer 115 may include an organic material. In an embodiment, for example, the first organic insulating layer 115 may include an organic insulating material including a general-purpose polymer, such as PMMA or PS, a polymer derivate having a phenol-based group, an acrylic polymer, an imide-based polymer, an aryl ether-based polymer, an amide-based polymer, a fluorine-based polymer, a p-xylene-based polymer, a vinyl alcohol-based polymer, and any blends thereof.

The second organic insulating layer 116 may include an organic material. The second organic insulating layer 116 may include an organic insulating material including a general-purpose polymer, such as PMMA or PS, a polymer derivate having a phenol-based group, an acrylic polymer, an imide-based polymer, an aryl ether-based polymer, an amide-based polymer, a fluorine-based polymer, a p-xylene-based polymer, a vinyl alcohol-based polymer, or any blends thereof.

The inorganic insulating layer IIL arranged in the plurality of base portions BSP may not be arranged in the plurality of bridge portions BRP in the sensor area SA. That is, the inorganic insulating layer IIL may be removed from the bridge portion BRP. Because the bridge portion BRP of the display panel 10 may be bent as described above, it is important for the bridge portion BRP to have flexibility. The inorganic insulating layer IIL has less flexibility than the organic insulating layer OIL and is particularly vulnerable to cracking. The cracking may provide a path through which moisture and oxygen penetrate into the first base portion BSP1 or the like. Accordingly, as shown in FIG. 8, as the inorganic insulating layer IIL is removed from the bridge portion BRP, the flexibility of the bridge portion BRP may be improved, and an occurrence of cracking may be significantly reduced.

The inorganic insulating layer IIL may define the open portion IIL-OP therein to correspond to the bridge portion BRP. The open portion IIL-OP may refer to a region from which the inorganic insulating layer IIL is removed. The upper surface of the substrate 100 may be exposed through the open portion IIL-OP. Although the region from which the inorganic insulating layer IIL is removed is defined as the open portion IIL-OP, the inorganic insulating layer IIL remaining on a plane may have an island shape corresponding to the first base portion BSP1. The open portion IIL-OP may extend from the bridge portion BRP toward the first base portion BSP1. That is, an area of the open portion IIL-OP may be greater than or equal to an area of the bridge portion BRP.

In an embodiment, the organic material layer OL may be arranged in the open portion IIL-OP of the inorganic insulating layer IIL. The organic material layer OL may be buried in the open portion IIL-OP of the inorganic insulating layer IIL and fill a step of the inorganic insulating layer IIL, which is formed by the open portion IIL-OP. The thickness of the organic material layer OL may be substantially equal to the height of the step of the inorganic insulating layer IIL, which is formed by the open portion IIL-OP. The flexibility of the bridge portion BRP may be improved by filling the open portion IIL-OP of the inorganic insulating layer IIL with the organic material layer OL.

In addition, the plurality of biosensors BS may be arranged over inorganic insulating layers IIL of the plurality of base portions BSP or organic material layers OL of the plurality of bridge portions BRP, respectively. In detail, the first biosensor BS1 may be arranged over the inorganic insulating layer IIL of the first base portion BSP1, and the second biosensors BS2 may be arranged over the organic material layer OL of the first bridge portion BRP1.

Each of the plurality of biosensors BS may include a first electrode E1, a second electrode E2, and first and second coating layers CL1 and CL2 connecting the first electrode E1 to the second electrode E2. In detail, the first biosensors BS1 arranged in the plurality of base portions BSP may each include a first coating layer CL1, and the second biosensors BS2 arranged in the plurality of bridge portions BRP may each include a second coating layer CL2.

The first electrode E1 and the second electrode E2 may detect a changed current value or resistance value of the M13 bacteriophage exposed to the target material. The first electrode E1 and the second electrode E2 may be arranged on substantially the same layer as the first source electrode SE1 (see FIG. 6) and the first drain electrode DE1 (see FIG. 6) of the driving thin-film transistor T1 arranged in the plurality of display portions DP (see FIG. 4A). In detail, the first biosensors BS1 arranged in the plurality of base portions BSP may be configured to use the sensor source electrode SE′ and the sensor drain electrode DE′ of the sensor thin-film transistor ST′ as the first electrode E1 and the second electrode E2, respectively. The second biosensors BS2 arranged in the plurality of bridge portions BRP may be arranged on the same layer as the sensor source electrode SE′ and the sensor drain electrode DE′ of the sensor thin-film transistor ST′, and metal layers spaced apart from each other may be used as the first electrode E1 and the second electrode E2.

Each of the first coating layer CL1 and thee second coating layer CL2 may include a genetically-modified M13 bacteriophage. The first and second coating layers CL1 and CL2 may be formed by coating an M13 bacteriophage-containing solution using a meniscus-dragging deposition (“MDD”) coating method. The first coating layer CL1 and the second coating layer CL2 form a convex meniscus, and M13 bacteriophages included in the first coating layer CL1 and the second coating layer CL2 may connect the first electrode E1 and the second electrode E2 to each other.

The first organic insulating layer 115 and the second organic insulating layer 116 may define a first opening OP1 and a second opening OP2 therein, which expose the plurality of biosensors BS to the outside. That is, the first and second openings OP1 and OP2 may pass through the first organic insulating layer 115 and the second organic insulating layer 116. In an embodiment, for example, in the plurality of base portions BSP, the first biosensor BS1 may be exposed to the outside through the first opening OP1. In the plurality of bridge portions BRP, the second biosensor BS2 may be exposed to the outside through the second opening OP2.

In detail, the first and second openings OP1 and OP2 may allow the coating layers CL1 and CL2 of the plurality of biosensors BS to be in contact with external air. In an embodiment, for example, the first opening OP1 of the organic insulating layer OIL allows the first coating layer CL1 of the first biosensor BS1 to be in contact with external air, such that the M13 bacteriophage included in the first coating layer CL1 may detect the target material. Similarly, the second opening OP2 of the organic insulating layer OIL allows the second coating layer CL2 of the second biosensor BS2 to be in contact with external air, such that the M13 bacteriophage included in the second coating layer CL2 may detect the target material.

Although the first and second coating layers CL1 and CL2 are desirable to be exposed through the first and second openings OP1 and OP2 of the organic insulating layer OIL, widths of the first and second openings OP1 and OP2 may be limited to prevent external contaminants from penetrating the display panel 10. In an embodiment, for example, the widths of the first and second openings OP1 and OP2 may be designed to expose only the first and second coating layers CL1 and CL2. That is, a width of the first opening OP1 may be equal to or less than a width of the first coating layer CL1. A width of the second opening OP2 may be equal to or less than a width of the second coating layer CL2.

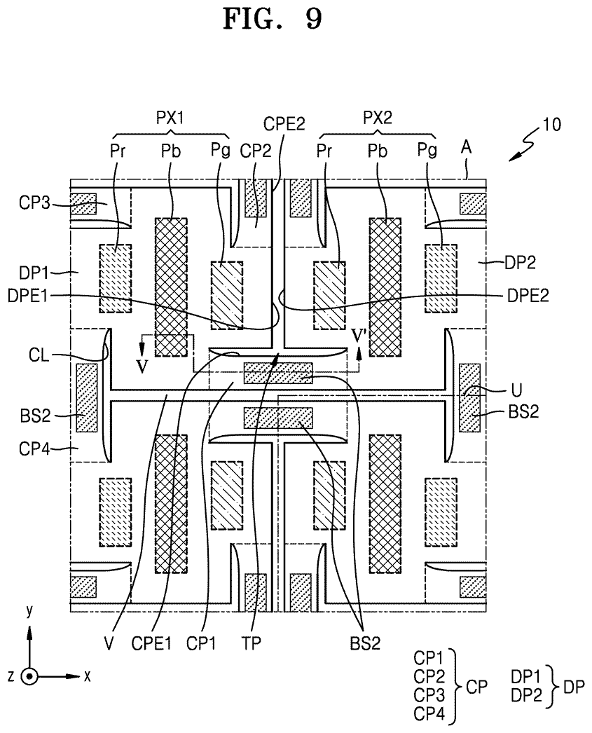

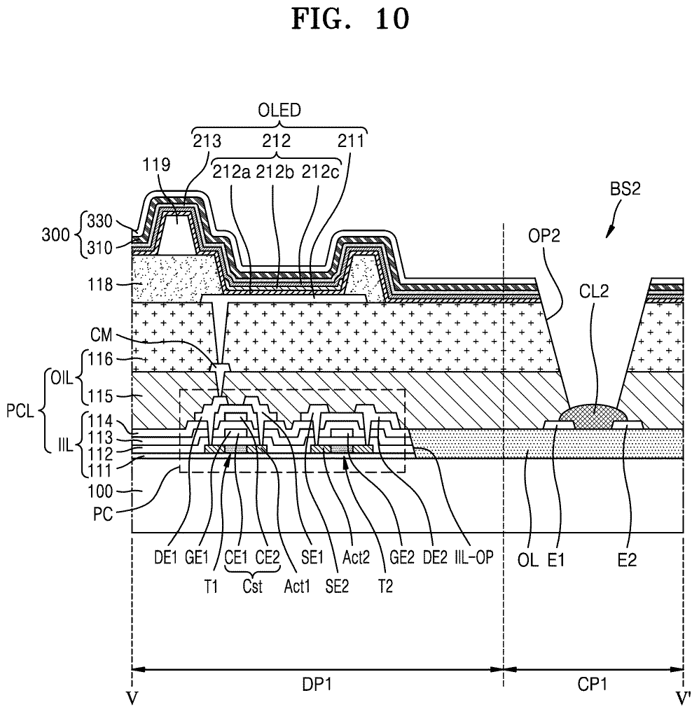

FIG. 9 is an enlarged plan view of a portion of the display apparatus, according to another embodiment, and FIG. 10 is a schematic cross-sectional view of the display panel 10 taken along line V-V′ of FIG. 9. Referring to FIGS. 9 and 10, except for features of the connection portion CP, other features are the same as those described with reference to FIGS. 4A to 8. The same reference numerals among the elements of FIGS. 9 and 10 are replaced with those previously described with reference to FIGS. 4A to 8, and hereinafter, differences are mainly described.

FIG. 9 is a schematic enlarged view of a region A of FIG. 2, i.e., a portion of the display area DA. Referring to FIG. 9, the display area DA may include the display portion DP and the connection portion CP, as described above. The display portion DP may include the first display portion DP1 and the second display portion DP2, and the connection portion CP may include the first connection portion CP1, the second connection portion CP2, the third connection portion CP3, and the fourth connection portion CP4. The plurality of display portions DP may be spaced apart from each other, and the connection portion CP may extend between the display portions DP adjacent to each other. In an embodiment, for example, the first display portion DP1 and the second display portion DP2 may be connected to each other by the first connection portion CP1, and the first display portion DP1, the second display portion DP2, and the first connection portion CP1 may be integrally provided as a single body.

In the case of the display apparatus according to another embodiment as shown in FIG. 9, the display area DA may include the plurality of biosensors BS. In this case, the plurality of biosensors BS may be arranged in the plurality of connection portions CP in the display area DA, respectively. That is, the biosensor BS arranged in the display area DA may be the second biosensor BS2 arranged in the bridge portion BRP of FIG. 7. In an embodiment, for example, each of the first connection portion CP1, the second connection portion CP2, the third connection portion CP3, and the fourth connection portion CP4 may include the second biosensor BS2.

The second biosensor BS2 may be an electrical sensor of which electrical resistance changes when exposed to the target material, as described above. The second biosensor BS2 may include an M13 bacteriophage.

Referring to FIG. 10, the substrate 100, the pixel circuit layer PCL, the organic light-emitting diode OLED, the encapsulation layer 300, and the plurality of biosensors BS may be arranged in the display area DA. In detail, the pixel circuit layer PCL may be arranged over the display portion DP of the display area DA, and the plurality of biosensors BS may be arranged over the connection portion CP of the display area DA.

The pixel circuit layer PCL may be arranged in the first display portion DP1. The pixel circuit layer PCL may include the driving thin-film transistor T1, the switching thin-film transistor T2, and the storage capacitor Cst. The pixel circuit layer PCL may include the inorganic insulating layer IIL and the organic insulating layer OIL arranged under and/or over the elements of the driving thin-film transistor T1. In addition, the inorganic insulating layer IIL arranged in the plurality of display portions DP may not be arranged over the connection portions CP of the display area DA. Accordingly, the inorganic insulating layer IIL may define the open portion IIL-OP therein corresponding to the connection portion CP, and the organic material layer OL may be arranged in the open portion IIL-OP of the inorganic insulating layer IIL.

The plurality of biosensors BS may be arranged over the plurality of connection portions CP, respectively. That is, the biosensors BS may not be arranged over the plurality of display portions DP in the display area DA, and the second biosensor BS2 may be arranged only over the plurality of connection portions CP in the display area DA.

In an embodiment, for example, the second biosensor BS2 may be arranged over the organic material layer OL of the first connection portion CP1.

The plurality of second biosensors BS2 arranged over the connection portions CP may each include the first electrode E1, the second electrode E2, and the second coating layer CL2 connecting the first electrode E1 to the second electrode E2, similar to the biosensors BS of FIG. 7. The first electrode E1 and the second electrode E2 may detect a changed current value or resistance value of the M13 bacteriophage exposed to the target material. The first electrode E1 and the second electrode E2 may be arranged on substantially the same layer as the first source electrode SE1 and the first drain electrode DE1 of the driving thin-film transistor T1 arranged in the plurality of display portions DP. The second coating layer CL2 may include a genetically-modified M13 bacteriophage. M13 bacteriophages included in the second coating layer CL2 may connect the first electrode E1 to the second electrode E2.

The first organic insulating layer 115 and the second organic insulating layer 116 may define a plurality of second openings OP2 therein, which expose the plurality of second biosensors BS2 to the outside. That is, the plurality of second openings OP2 may pass through the first organic insulating layer 115 and the second organic insulating layer 116. Accordingly, the plurality of second openings OP2 may allow the second coating layer CL2 of the plurality of second biosensors BS2 to be in contact with external air. That is, the second opening OP2 of the organic insulating layer OIL allows the second coating layer CL2 of the second biosensors BS2 to be in contact with external air, such that the M13 bacteriophage included in the second coating layer CL2 may detect the target material.

In addition, though not shown in FIG. 10, the connection wire CWL (see FIG. 6) may be arranged over the organic material layer OL. The connection wire CWL (see FIG. 6) may be arranged in the connection portion CP to electrically connect the pixel circuit PC of the first display portion DP1 to a pixel circuit of another adjacent display portion (e.g., the pixel circuit of the second display portion DP2). However, the connection wire CWL may be arranged in a region of the connection portion CP where the second biosensor BS2 is not arranged.

The organic light-emitting diode OLED may be disposed on the organic insulating layer OIL. The organic light-emitting diode OLED may include the pixel electrode 211, the intermediate layer 212, and the opposite electrode 213. The organic light-emitting diode OLED may be arranged in each of the plurality of display portions DP. In this case, a plurality of pixel electrodes 211 may be arranged in the display portions DP to be spaced apart from each other, respectively, but the intermediate layer 212 and the opposite electrode 213 may be integrally formed as a single body over the entire display area DA. Accordingly, the intermediate layer 212 and the opposite electrode 213 may be arranged not only over the display portion DP but also over the connection portion CP.

However, the intermediate layer 212 and the opposite electrode 213 define an opening therein overlapping the second opening OP2 of the organic insulating layer OIL and may expose the second coating layer CL2 of the second biosensor BS2. The opening of the intermediate layer 212 and the opposite electrode 213 may have a width equal to or less than a width of the second coating layer CL2 to expose only the second coating layer CL2.

The encapsulation layer 300 may be arranged on the opposing electrode 213. In some embodiments, the encapsulation layer 300 may include at least one inorganic encapsulation layer. In an embodiment, for example, the encapsulation layer 300 may include the first inorganic encapsulation layer 310 and the second inorganic encapsulation layer 330, which are sequentially stacked as in FIG. 10. The encapsulation layer 300 may be integrally formed as a single body over the entre display area DA, similar to the intermediate layer 212 and the opposite electrode 213. Accordingly, the encapsulation layer 300 may be arranged not only over the display portion DP but also over the connection portion CP. However, the embodiment is not limited thereto, and in another embodiment, the encapsulation layer 300 may be arranged only over the plurality of display portions DP and removed from the plurality of connection portions CP.

When the encapsulation layer 300 is formed over the display portion DP, however, the encapsulation layer 300 may define an opening therein overlapping the second opening OP2 of the organic insulating layer OIL. The opening of the encapsulation layer 300 may expose the second coating layer CL2 of the second biosensor BS2. The opening of the encapsulation layer 300 may have a width equal to or less than the width of the second coating layer CL2 to expose only the second coating layer CL2.

FIGS. 11A to 11E are diagrams for describing an M13 bacteriophage of a biosensor, according to an embodiment. FIG. 11A is a diagram illustrating that M13 bacteriophages have a smectic spiral nanofiber structure. FIG. 11B is an enlarged view illustrating that the M13 bacteriophages of FIG. 11A each has a size of 1 micrometer (μm). FIG. 11C is a diagram of one fiber in a smectic nanofiber bundle of M13 bacteriophages. FIG. 11D is an enlarged view of a portion of a surface protein of one M13 bacteriophage. FIG. 11E is a graph illustrating an amount of change in a current value of an M13 bacteriophage exposed to a target material.

Referring to FIGS. 11A to 11C, the plurality of biosensor BS (see FIG. 7) may each include an M13 bacteriophage VNP. The M13 bacteriophage VNP may include tubular nanoparticles each having a length of about 880 nanometers (nm) and a diameter of about 6.6 nm. Because the M13 bacteriophage VNP includes protein particles expressed through a certain gene, sizes, shapes, and distributions thereof may be constant. That is, almost all M13 bacteriophages VNP may have substantially the same size and shape. Each M13 bacteriophage VNP may include, at opposite ends thereof, about 2700 pairs of proteins (protein VIII, hereinafter, referred to as pVIII), and 4 to 5 pairs of proteins (pIII, pVI, pVII, and pIX).

In addition, the M13 bacteriophage VNP used herein may include a case in which a protein containing an amino acid sequence is included in pVIII, which is a main surface protein of the M13 bacteriophage VNP.

The M13 bacteriophages VNP included in the plurality of biosensors BS, respectively, may have a smectic spiral nanofiber structure. In an embodiment, for example, the plurality of M13 bacteriophages VNP are arranged in a smectic structure, and bundles of M13 bacteriophages VNP are spirally pulled to have a smectic spiral nanofiber structure.

Referring to FIGS. 11D and 11E, the plurality of biosensors BS may be electrical sensors in which, when exposed to the target material, surface proteins of the M13 bacteriophages VNP swell, such that an electron movement path changes. The M13 bacteriophage VNP has a dynamic transformation property of expanding or contracting according to changes in an external environment. As in FIG. 11D, when the M13 bacteriophage VNP is exposed to the target material, a surface protein thereof swell, such that a thickness of the surface protein may change. As the thickness of the surface protein change, a movement path of electrons transferred through the M13 bacteriophage VNP also changes, such that a current value and resistance value detected by the M13 bacteriophage VNP also change.

FIG. 11E is a graph illustrating an amount of change in a current value of the M13 bacteriophage VNP exposed to the target material. A horizontal axis of the present graph represents a duration of an experiment in units of seconds (sec). A vertical axis of the present graph represents a current value detected by the M13 bacteriophage VNP in units of microamperes (μA). In the present experiment, 2,000 seconds after the start of measuring the current value of the M13 bacteriophage VNP, the target material, ethanol, was exposed to the M13 bacteriophage VNP. Referring to FIG. 11E, it may be seen that, when ethanol is exposed to the M13 bacteriophage VNP, the current value, which was only at a level of about 10 μA to about 30 μA, has rapidly changed to a level of about 300 μA to about 500 μA. That is, the biosensors BS including the M13 bacteriophage VNP may be used as electrical sensors capable of detecting the target material.

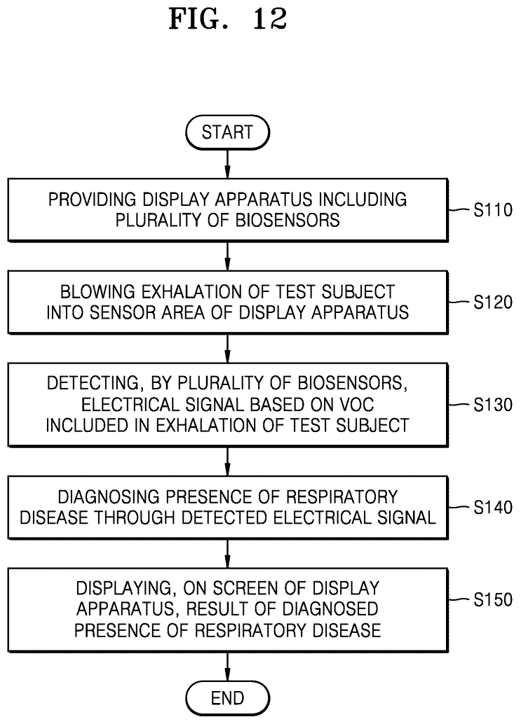

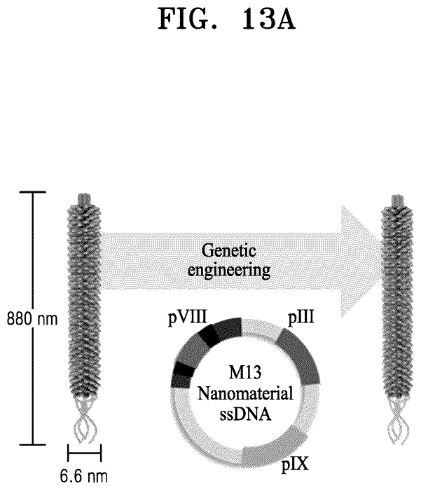

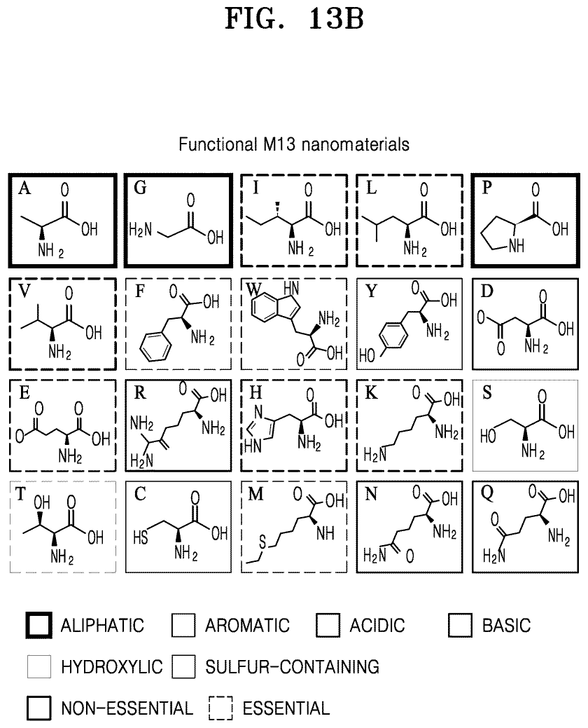

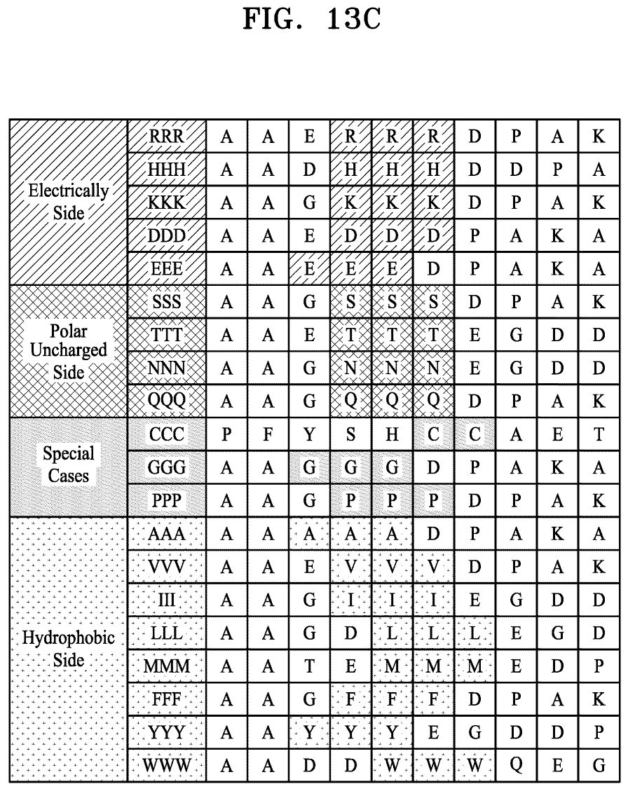

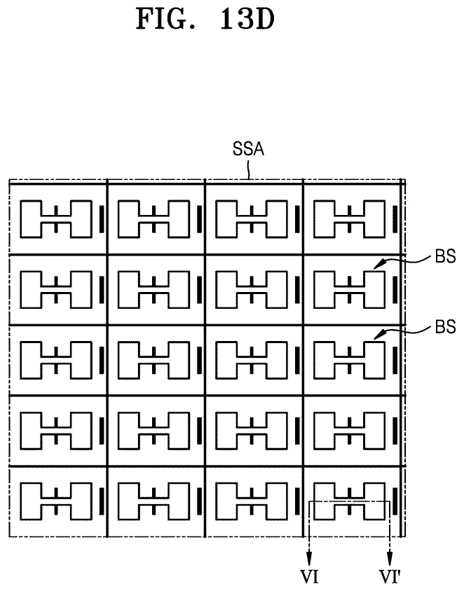

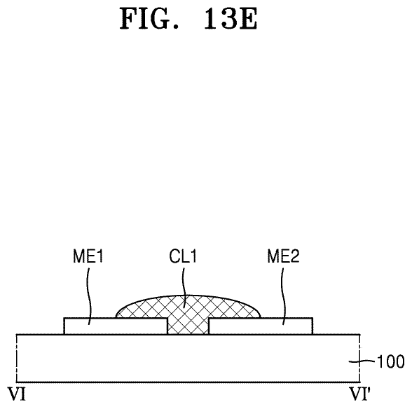

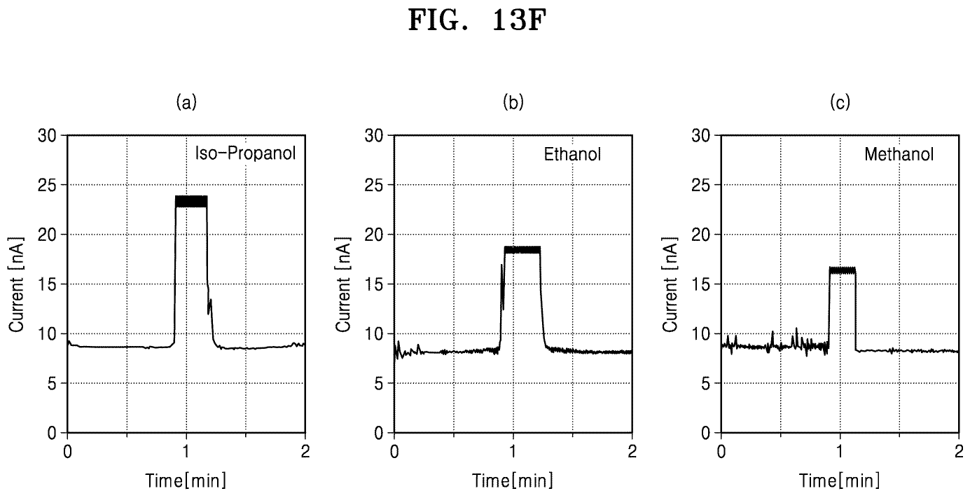

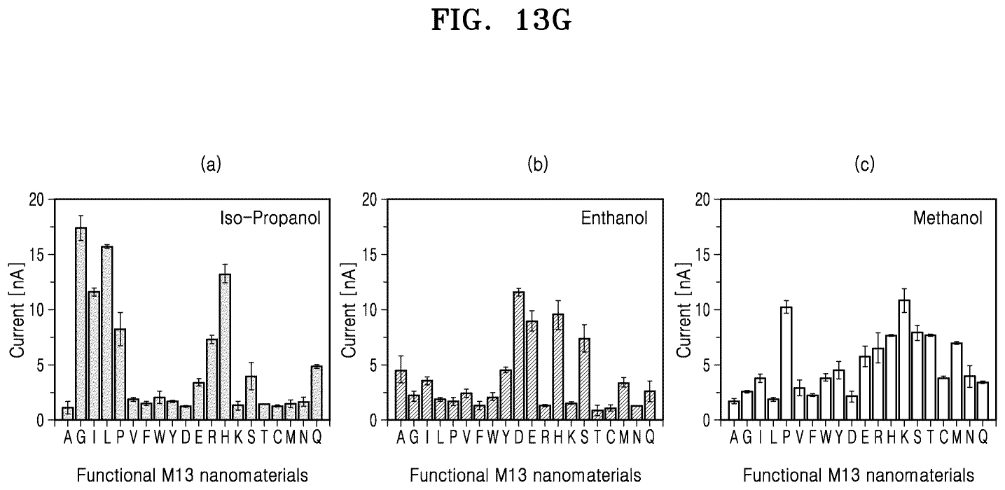

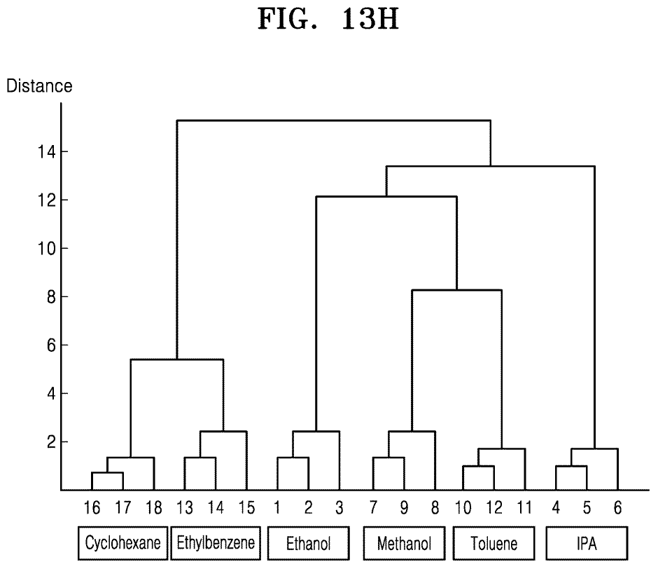

FIG. 12 is a flowchart of a sensing method using a display apparatus, according to an embodiment, and FIGS. 13A to 13G are diagrams for describing a sensing method using a biosensor, according to an embodiment.

Referring to FIG. 12, the sensing method using the display apparatus, according to an embodiment, may include providing a display apparatus including a plurality of biosensors (S110), blowing an exhalation of a test subject into a sensor area of the display apparatus (S120), detecting, by the plurality of biosensors, an electrical signal based on a volatile organic compound (VOC) included in the exhalation of the test subject (S130), diagnosing whether a respiratory disease is present through the detected electrical signal (S140), and displaying the diagnosed result, on a screen of the display apparatus (S150). However, the sensing method using the display apparatus is not limited to the above example, and at least a part of each operation may be omitted or the sensing method may further include at least another operation with reference to other descriptions of the present specification.

First, the display apparatus 1 may include a plurality of biosensors BS using an M13 bacteriophage (S110).

Referring to FIG. 13A, the M13 bacteriophage included in the plurality of biosensors BS may be a genetically-modified bacteriophage. The genetically-modified bacteriophage may be prepared by amplifying a phage by expressing a peptide having a desired sequence on a surface protein of the M13 bacteriophage using a reverse transcription polymerase chain reaction.

A peptide having a specific sequence included in the surface protein of the M13 bacteriophage may be used as a reaction receptor that reacts to the target material. That is, a peptide of a modified M13 bacteriophage may react only with a specific VOC, such that the plurality of biosensors BS may selectively detect the target material. In an embodiment, for example, the target material reacting with the modified M13 bacteriophage may include ethanol, methanol, toluene, isopropanol (“IPA”), cyclohexane, ethylbenzene, or the like.

Referring to FIGS. 13B and 13C, M13 bacteriophages included in the plurality of biosensors BS, respectively, may be genetically modified differently from each other. In an embodiment, the plurality of biosensors BS may include 20 types of M13 bacteriophages genetically modified differently from each other, respectively. In detail, a surface protein of the genetically-modified M13 bacteriophage may include a peptide having a sequence in which at least one of 20 types of amino acids is consecutively arranged.

FIG. 13B is a table in which 20 types of amino acids are listed. The amino acids may be classified into aliphatic amino acids, aromatic amino acids, acidic amino acids, basic amino acids, hydrophilic amino acids, and sulfur-containing amino acids. The aliphatic amino acids may include alanine (A), glycine (G), isoleucine (I), leucine (L), proline (P), and valine (V). The aromatic amino acids may include phenylalanine (F), tryptophan (W), and tyrosine (Y). The acidic amino acids may include aspartic acid (D) and glutamic acid (E). The basic amino acids may include arginine (R), histidine (H), and lysine (K). The hydrophilic amino acids may include serine (S), threonine (T), asparagine (N), and glutamine (Q). The sulfur-containing amino acids may include cysteine (C) and methionine (M).

FIG. 13C is a table in which an amino acid sequence of 20 types of M13 bacteriophages genetically modified differently from each other is listed. That is, a surface protein of the genetically-modified M13 bacteriophage may include a peptide having a sequence in which at least one of the 20 types of amino acids as described above is consecutively arranged. In an embodiment, some of the 20 types of genetically-modified M13 bacteriophages may include a polar-charged surface protein. In an embodiment, for example, an M13 bacteriophage including the polar-charged surface protein may be formed by expressing a peptide having a sequence in which at least one of R, H, K, D, and E is consecutively arranged. Another portions of the 20 types of genetically-modified M13 bacteriophages may include a polar-uncharged surface protein. In an embodiment, for example, an M13 bacteriophage including the polar-uncharged surface protein may be formed by expressing a peptide having a sequence in which at least one of S, T, N, and Q is consecutively arranged. Another portions of the 20 types of genetically-modified M13 bacteriophages may include a hydrophobic surface protein. In an embodiment, for example, an M13 bacteriophage including the hydrophobic surface protein may be formed by expressing a peptide having a sequence in which at least one of A, V, I, L, M, F, Y, and W is consecutively arranged. Others of the 20 types of genetically-modified M13 bacteriophages may be formed by expressing a peptide having a sequence in which at least one of C, G, and P is consecutively arranged. As listed in FIG. 13C, the plurality of biosensors BS may each include the 20 types of genetically-modified M13 bacteriophages. Accordingly, there may be 20 types of the plurality of biosensors BS.

Referring to FIGS. 13 and 13E, the display apparatus according to an embodiment may include a sensor array SSA. The sensor array SSA may be defined as a unit of sensors, in which the plurality of biosensors BS are integrated. In an embodiment, the sensor area SA may include a plurality of sensor arrays SSA, and one sensor array SSA may include all 20 different types of biosensors BS. In an embodiment, for example, the sensor array SSA may include all 20 types of M13 bacteriophages formed by expressing a peptide having a sequence listed in FIG. 13C.

As described above, each of the plurality of biosensors BS included in the sensor array SSA may include a first electrode ME1, a second electrode ME2, and the first coating layer CL1 (or the second coating layer CL2) connecting the first electrode ME1 to the second electrode ME2. Each of the first and second coating layers CL1 and CL2 may include one of 20 types of M13 bacteriophages genetically modified differently from each other.

However, an arrangement of the plurality of biosensors BS constituting the sensor array SSA is not limited to being arranged in a row as shown in FIG. 13D. As shown in FIG. 7, some of the plurality of biosensors BS constituting the sensor array SSA may be arranged in the plurality of base portions BSP (see FIG. 7) of the sensor area SA, respectively, and others of the plurality of biosensors BS may be arranged in the plurality of bridge portions BRP (see FIG. 7) of the sensor area SA, respectively.

Next, an exhalation of a test subject may be blown into the sensor area of the display apparatus (S120).

The display apparatus 1 including the plurality of biosensors BS may sense a target material in a gas state, emitted from a user, i.e., the test subject, and display a diagnosis result of a disease that the user suffers from the target material. In detail, target materials reacting with the plurality of biosensors BS are VOCs included in respiratory by-products of a patient with respiratory disease. The aforementioned target material is not discharged in exhalations of normal people, but in the case of the patient with respiratory disease, the aforementioned target material is discharged through respiration. That is, as the exhalation of the test subject is blown into the display apparatus 1, it may be tested whether the VOCs included in the respiratory by-products of the patient with respiratory disease are included.

Next, the plurality of biosensors BS may detect an electrical signal based on a VOC included in the exhalation of the test subject (S130). The detecting of the electrical signal (S130) may include detecting, by a genetically-modified M13 bacteriophage, a VOC included in the exhalation (S130-1), allowing an electron movement path to change due to swelling of a surface protein of the M13 bacteriophage in response to the VOC (S130-2), and calculating a changed electrical signal of each of the plurality of biosensors BS (S130-3).

Referring to FIGS. 13F and 13G, as described above, the biosensor BS including the M13 bacteriophage may be an electrical sensor of which electrical signal data changes in response to a target material included in the respiratory by-products of the patient with respiratory disease. That is, when the M13 bacteriophage is exposed to the target material, a surface protein swells, and the electron movement path changes. Accordingly, a current value or resistance value detected through the biosensor BS may change. The target material that is detectable by the M13 bacteriophage may include ethanol, methanol, toluene, isopropanol (IPA), cyclohexane, ethylbenzene, and/or the like.

Graphs (a), (b), and (c) of FIG. 13F are results of changes in current values of the biosensor BS exposed to isopropanol (IPA), ethanol, and methanol, respectively. The biosensor BS has a current value at a level of 9 nA when not exposed to the target material, but when exposed to IPA, the current value has greatly changed to a level of 23 nA. Also, when the biosensor BS is exposed to ethanol, the current value has greatly changed to a level of 18 nA, and when exposed to methanol, the current value has greatly changed to a level of 16 nA. Accordingly, it may be seen that, when the biosensor BS is exposed to the target material, such as IPA, ethanol, and methanol, the electrical signal data of the M13 bacteriophage has rapidly changed.

Graphs (a), (b), and (c) of FIG. 13G are results of current values of the sensor array SSA exposed to IPA, ethanol, and methanol, respectively. That is, when each of 20 types of biosensors BS included in the sensor array SSA is exposed to the target material, each of the biosensors BS may have a different value. In an embodiment, for example, when IPA is exposed to the sensor array SSA, the biosensor BS including an M13 bacteriophage in which a peptide having a sequence in which A is consecutively arranged is expressed may have a current value at a level of 2 nA. In contrast, the biosensor BS including an M13 bacteriophage in which a peptide having a sequence in which G is consecutively arranged is expressed may have a current value at a level of 15 nA. As described above, the 20 types of biosensors BS may have different current values for the same target material.

As a result, when the exhalation of the test subject is blown into the sensor array SSA, each of the biosensors BS may detect an electrical signal based on the VOC included in the exhalation. Because the electrical signal data is differently detected according to a type of genetically-modified M13 bacteriophage, the presence of a target material may be more accurately determined in a subsequent sensing operation.

Next, the presence of a respiratory disease may be diagnosed through the detected electrical signal (S140). The diagnosing of the presence of the respiratory disease (S140) may include accumulating data about an electrical signal for each VOC (S140-1), determining a type of the VOC by comparing the electrical signal for the VOC included in the exhalation of the test subject with the accumulated data (S140-2), and determining the presence of a respiratory disease according to the type of the VOC included in the exhalation of the test subject (S140-3).

The accumulating of the data about the electrical signal for each VOC (S140-1) may be performed by a statistical analysis method, such as hierarchical clustering analysis (HCA). First, by exposing target materials to the sensor array SSA by type, electrical signal data for each target material may be obtained. In this case, the electrical signal data for each target material may include electrical signal data based on changes in current value or changes in resistance value of each of the target materials.

Thereafter, the measured electrical signal data for each target material may be accumulated and stored through HCA. The HCA is a statistical analysis method of obtaining Euclidean distances of pieces of measured data, forming clusters from values having the closest physical Euclidean distances, and finally forming all data into one cluster. Also, such a clustering of pieces of data may be visualized as a dendrogram. FIG. 13H is a graph illustrating electrical signal data for each VOC analyzed through HCA and represented as a dendrogram. Accordingly, it may be identified that similar electrical signal data is detected for each VOC included in the exhalation, and it may be identified that electrical signal data measured by the biosensor BS using the M13 bacteriophage has reproducibility.

In the determining of the type of the VOC (S140-2), the type of the VOC may be identified by the dendrogram analyzed through the HCA. As described above with reference to FIG. 13F, the accumulated electrical signal data for each VOC may be visualized as a dendrogram through the HCA. Then, the exhalation of the test subject is blown into the display apparatus 1, and electrical signal data for the VOC included in the exhalation of the test subject is compared with the previously analyzed dendrogram, such that the type of the VOC may be easily identified.

As a result, by comparing the accumulated electrical signal data for each target material with the electrical signal data for the exhalation of the test subject, the VOC detected by the biosensor BS among the by-products included in the exhalation of the test subject may be identified as one of the target materials. That is, the presence of a respiratory disease in the test subject may be diagnosed according to whether the VOC included in the exhalation of the test subject matches a respiratory disease-related target material.

Finally, a result of the diagnosis may be displayed on a screen of the display apparatus 1 (S150).

The result of the presence of the respiratory disease diagnosed through operations S110 to S140 may be displayed as an image through the display area DA of the display apparatus 1. The display apparatus 1 including the plurality of biosensors BS may display details of the diagnosis of the respiratory disease as described above as an image, and may be provided as a respiratory disease diagnosis sensor which enables intuitive determination with the naked eye using a simple method.

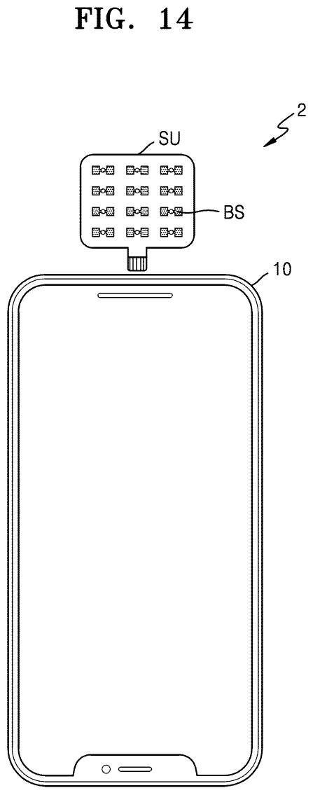

FIG. 14 is a schematic diagram of an electronic apparatus according to another embodiment. Referring to FIG. 14, except for features of a sensor unit SU, other features are the same as those described with reference to FIGS. 4A to 8. The same reference numerals among elements of FIG. 14 are replaced with those previously described with reference to FIGS. 4A to 8, and hereinafter, differences are mainly described.

Referring to FIG. 14, an electronic apparatus 2 may include a display panel 10 that displays an image, and the sensor unit SU of which electrical resistance changes when exposed to a target material. The sensor unit SU may include a plurality of biosensors BS. In detail, as described with reference to FIGS. 7 and 8, the sensor unit SU may include a substrate, the plurality of biosensors BS disposed on the substrate, and an insulating layer defining a plurality of openings therein, which expose the plurality of biosensors BS to the outside. Also, each of the plurality of biosensors BS may include a genetically-modified M13 bacteriophage.

However, the sensor unit SU of FIG. 14 may be detached from the display panel 10. The sensor unit SU may be a separate external module electrically connected to the display panel 10. That is, the sensor unit SU using the M13 bacteriophage may be selectively detached not only from a specific user's electronic apparatus but also from another user's electronic apparatus. Accordingly, the electronic apparatus according to another embodiment may easily diagnose the presence of a respiratory disease by using the M13 bacteriophage, and may also promote convenience in diagnosis work as the sensor unit SU is detachable.

As described above, the disclosure has been described with reference to the one or more embodiments shown in the accompanying drawings, but should be considered in a descriptive sense only. Those of ordinary skill in the art will understand that various modifications and changes to the embodiments may be made therefrom. Therefore, the true technical scope of protection of the disclosure should be defined by the technical spirit of the appended claims.

The display apparatus according to the one or more embodiments as described above may easily diagnose the presence of a respiratory disease by using the M13 bacteriophage. Also, according to the one or more embodiments, a flexible and stretchable display apparatus may be provided. However, the aforementioned effects are examples, and do not limit the scope of the disclosure.

It should be understood that the embodiments described herein should be considered in a descriptive sense only and not for purposes of limitation. Descriptions of features or aspects within each of the embodiments should typically be considered as available for other similar features or aspects in other embodiments. While one or more embodiments have been described with reference to the figures, it will be understood by one of ordinary skill in the art that various changes in form and details may be made therein without departing from the spirit and scope as defined by the following claims.

What is claimed is:

1. A display apparatus comprising:
a substrate including a sensor area and a display area;
a plurality of biosensors disposed on the substrate, and of which electrical resistance changes when exposed to a target material; and
an organic insulating layer defining a plurality of openings therein, which expose the plurality of biosensors to an outside,
wherein the display area includes a plurality of first through-portions, and a plurality of display portions spaced apart from each other by the plurality of first through-portions, and
the sensor area includes a plurality of second through-portions, and a plurality of base portions spaced apart from each other by the plurality of second through-portions.

2. The display apparatus of claim 1, wherein each of the plurality of biosensors includes a first electrode, a second electrode, and a coating layer, which connects the first electrode to the second electrode.

3. The display apparatus of claim 2, wherein the first electrode and the second electrode are arranged on substantially a same layer as a source electrode and a drain electrode of a thin-film transistor arranged in the plurality of display portions.

4. The display apparatus of claim 2, wherein the plurality of openings allow the coating layer to be in contact with external air.

5. The display apparatus of claim 2, wherein the coating layer includes an M13 bacteriophage.

6. The display apparatus of claim 5, wherein the plurality of biosensors each are an electrical sensor in which, when exposed to the target material, a surface protein of the M13 bacteriophage swells and an electron movement path changes.

7. The display apparatus of claim 5, wherein the M13 bacteriophage is provided in plurality, and the plurality of biosensors include the M13 bacteriophages genetically modified differently from each other, respectively.

8. The display apparatus of claim 7, wherein a surface protein of each of the genetically-modified M13 bacteriophages includes a peptide having a sequence in which at least one of amino acids is consecutively arranged.

9. The display apparatus of claim 1, wherein the display area further includes a plurality of connection portions extending in different directions from the plurality of display portions,
the sensor area further includes a plurality of bridge portions extending in different directions from the plurality of base portions,
at least one of the plurality of connection portions is configured to connect two adjacent display portions among the plurality of display portions to each other, and
at least one of the plurality of bridge portions is configured to connect two adjacent base portions among the plurality of base portions to each other.

10. The display apparatus of claim 9, wherein a distance between the two adjacent display portions and a distance between the two adjacent base portions are changed by an external force, and
the plurality of connection portions and the plurality of bridge portions are stretchable by the external force.

11. The display apparatus of claim 9, wherein the plurality of biosensors include:
a plurality of first biosensors arranged in the plurality of base portions, respectively; and
a plurality of second biosensors arranged in the plurality of bridge portions, respectively.

12. The display apparatus of claim 11, wherein each of the plurality of base portions includes a sensor thin-film transistor, and
a source electrode and a drain electrode of the sensor thin-film transistor are used as metal electrodes of the plurality of first biosensors.

13. The display apparatus of claim 11, wherein each of the plurality of bridge portions has a structure in which the substrate, an organic material layer disposed on the substrate, and the plurality of second biosensors disposed on the organic material layer are stacked.

14. The display apparatus of claim 9, wherein the plurality of biosensors are arranged in the plurality of connection portions of the display area, respectively.

15. The display apparatus of claim 1, wherein the substrate further includes a peripheral area surrounding the display area, and
the sensor area overlaps the peripheral area.

16. An electronic apparatus comprising a sensor unit, wherein the sensor unit includes:
a substrate;
a plurality of biosensors disposed on the substrate, wherein each of the plurality of biosensors includes a first electrode, a second electrode, and a coating layer connecting the first electrode to the second electrode; and
an organic insulating layer defining a plurality of openings therein, which expose the plurality of biosensors to an outside,
wherein the coating layer includes an M13 bacteriophage, and
when the sensor unit is exposed to a target material, electrical resistance of the plurality of biosensors changes.

17. The electronic apparatus of claim 16, further comprising a display panel, wherein the sensor unit is a separable external module electrically connected to the display panel for displaying an image, and the sensor unit is detachable from the display panel.

18. The electronic apparatus of claim 16, further comprising a display panel for displaying an image,
wherein a sensor area and a display area are defined in the display panel, and
the sensor unit is arranged in the sensor area.

19. The electronic apparatus of claim 18, wherein the display area includes a plurality of first through-portions, a plurality of display portions spaced apart from each other by the plurality of first through-portions, and a plurality of connection portions extending in different directions from the plurality of display portions,
the sensor area includes a plurality of second through-portions, a plurality of base portions spaced apart from each other by the plurality of second through-portions, and a plurality of bridge portions extending in different directions from the plurality of base portions,
at least one of the plurality of connection portions is configured to connect two adjacent display portions among the plurality of display portions to each other, and
at least one of the plurality of bridge portions is configured to connect two adjacent base portions among the plurality of base portions to each other.

20. The electronic apparatus of claim 19, wherein the plurality of biosensors include:
a plurality of first biosensors arranged in the plurality of base portions, respectively; and
a plurality of second biosensors arranged in the plurality of bridge portions, respectively.

21. The electronic apparatus of claim 19, wherein the first electrode and the second electrode are arranged on substantially a same layer as a source electrode and a drain electrode of a thin-film transistor arranged in the plurality of display portions.

22. A sensing method of a display apparatus, the sensing method comprising:
providing a display apparatus including a substrate, a plurality of biosensors, and an organic insulating layer, the substrate including a sensor area and a display area, the plurality of biosensors disposed on the substrate and of which electrical resistance changes when exposed to a target material, and the organic insulating layer defining a plurality of openings therein, which expose the plurality of biosensors to the outside;
blowing an exhalation of a test subject into the sensor area;
detecting, by the plurality of biosensors, an electrical signal based on a volatile organic compound (VOC) included in the exhalation of the test subject; and
diagnosing whether a respiratory disease is present through the detected electrical signal.

23. The sensing method of claim 22, wherein the providing of the display apparatus further comprises providing a sensor array with the plurality of biosensors, each including an M13 bacteriophage genetically modified differently.

24. The sensing method of claim 23, wherein the detecting of the electrical signal comprises:
detecting, by the genetically-modified M13 bacteriophage, the VOC included in the exhalation;
allowing an electron movement path to change due to swelling of a surface protein of the M13 bacteriophage in response to the VOC; and
calculating a changed electrical signal of each of the plurality of biosensors.

25. The sensing method of claim 24, wherein the diagnosing of the presence of the respiratory disease comprises:
accumulating data about an electrical signal for each VOC;
determining a type of the VOC by comparing the electrical signal for the VOC included in the exhalation of the test subject with the accumulated data; and
determining whether the respiratory disease is present according to the type of the VOC included in the exhalation of the test subject.

26. The sensing method of claim 25, wherein the accumulating of the data about the electrical signal for each VOC is performed by a statistical analysis method, such as hierarchical clustering analysis (HCA).

27. The sensing method of claim 26, wherein, in the determining of the type of the VOC, the type of the VOC is identified by a dendrogram analyzed through the HCA.

28. The sensing method of claim 22, further comprising displaying, on a screen of the display apparatus, a result of the diagnosis.
